(19) United States
(12) Patent Application Publication
(10) Pub. No.: US 2025/0136636 A1
(43) Pub. Date: May 1, 2025

(54) NUCLEOTIDE COMPLEXES CAPABLE OF AN IMPROVED DNA SYNTHESIS YIELD

(71) Applicant: TOUCHLIGHT IP LIMITED, Hampton (GB)

(72) Inventors: Daniel KYSH, Hampton (GB); Paul ROTHWELL, Hampton (GB)

(73) Assignee: TOUCHLIGHT IP LIMITED, Hampton (GB)

(21) Appl. No.: 18/684,090

(22) PCT Filed: Aug. 16, 2022
(86) PCT No.: PCT/GB2022/052132

§ 371 Date: Feb. 15, 2024

(30) Foreign Application Priority Data
Aug. 16, 2021 (GB) 2111742.9

(51) Int. Cl. C07H 23/00; C12P 19/34

(52) U.S. Cl. CPC C07H 23/00; C12P 19/34

(57) ABSTRACT

The present invention relates to novel nucleotide complexes which enhance the enzymatic production of DNA. The nucleotide complexes includes a divalent cation at a ratio of 0.2-1.5 divalent cations per nucleotide, whilst monovalent ions (other than hydrogen or hydronium ions from any solvent) are minimal. The nucleotide complexes have desirable properties for the synthesis of deoxyribonucleic acid (DNA), in particular cell-free enzymatic synthesis of DNA, preferably on a large or industrial scale. Further, the invention includes improved processes for preparing said nucleotide complexes. Further complexes are disclosed which possess unique properties in accelerating DNA synthesis at higher concentrations, these complexes also containing zwitterion molecules.

### FIELD

The present invention relates to novel nucleotide complexes which enhance the enzymatic production of DNA. The nucleotide complexes include a divalent cation which is present at a ratio of 0.2-1.5 divalent cations per nucleotide, whilst monovalent ions (other than hydrogen or hydronium ions from any solvent) are minimal. The nucleotide complexes have desirable properties for the synthesis of deoxyribonucleic acid (DNA), in particular cell-free enzymatic synthesis of DNA, preferably on a large or industrial scale, with an improved yield and/or with an improved efficiency. Further, the invention includes improved processes for preparing said nucleotide complexes. The processes results in unique nucleotide complexes, wherein the complex is counter-ioned merely with a divalent cation at a ratio of 0.2-1.5 divalent cations per nucleotide. Substantially all of the moiety providing the monovalent cation from the starting material is removed. Further complexes are disclosed which possess unique properties in accelerating DNA synthesis at higher concentrations, these complexes also containing zwitterionic molecules.

### BACKGROUND

Amplification of deoxyribonucleic acid (DNA) may be carried out through use of cell-based processes, such as by culture of bacteria propagating DNA to be amplified in fermenters. Cell-free enzymatic processes for amplification of DNA from a starting template have also been described, including the polymerase chain reaction and strand-displacement reactions.

In the past, amplification of DNA on a test scale has been performed using apparatus based on microtitre plates and robotically controlled pipettes to add reaction components as required. Such apparatus and processes are suitable for manufacturing small quantities of DNA for test purposes but do not provide sufficient quantities for other purposes. Large-scale amplification and manufacture of specific nucleic acids and proteins has mostly been carried out through cell-based processes. Such methods are generally effective for production of very large volumes of product but costly to set up. Further, it is preferable to synthesise DNA in a cell-free environment for clinical and therapeutic purposes. In terms of amplification of plasmid using routine methods in the art such as fermentation, a commercial scale operation may be able to manufacture 2.6 g/l. This is considered by those in the art to be at “industrial scale”.

To date, biological catalysts such as polymerase have not been routinely exploited for industrial scale manufacture of DNA products in vitro and reactions have largely been limited to volumes at microliter scale. Scaling up processes using enzymatic synthesis of DNA has proved problematic, not least with the disappointing yield of DNA product.

The present applicants have previously addressed the ability of scaling-up using commercially available nucleotides. A new process was developed that involved adding fresh nucleotides to the reaction mixture as they became depleted or as the concentration of the product reached a threshold, as described in WO2016/034849, incorporated herein by reference. However, it was established that even higher yields could be achieved and the inventors have developed nucleotide complexes which are associated with a mixture of divalent cations and monovalent cations (PCT/GB2021/050366). It is a further improvement of the nucleotide complexes that is included in the present application.

Enzymatic DNA synthesis generally requires the use of a polymerase or polymerase-like enzyme to catalyse the addition of nucleotides to a nascent nucleic acid chain. Generally, a template DNA is required which is amplified in the reaction. However, it is also possible to perform template-free DNA synthesis, where the incorporation happens de novo.

It is important to note that due to the highly charged nature of nucleic acids, they are constantly surrounded by counter-ions to neutralise most of their charges which lessen the electrostatic repulsion between sections of sequence, so they can be condensed into neat, compact structures in the cell. The building blocks of nucleic acids, the nucleotides, are also an ionic species and require the presence of positive counter-ions in order to maintain electrical neutrality. Most, if not all, nucleotides are thus supplied as salts with a positive counter-ion. Without a positive counter-ion from a salt, nucleotides are presented as their free acid form where electrical neutrality is maintained by a hydrogen ion. Since the nucleotide has four negative charges, salts are typically prepared with 2 divalent cations, or 4 monovalent cations. It will be apparent to those skilled in the art that as soon as a nucleotide (salt or acid) is dispersed in water or other solvent, they may dissociate in solution into anionic and cationic components.

In general, nucleotides are supplied for DNA synthesis, amplification or sequencing as either a lithium or sodium salt. Lithium is generally preferred since these salts offer greater solubility and also stability to repeated freezing and thawing cycles than sodium salts and remain sterile due to the bacteriostatic activity of lithium towards various microorganisms, giving greater reliability and an extended shelf life. The use of these salts is so routine that those skilled in the art do not appear to question the counter-ion present with the nucleotide. Indeed, all of the nucleotides used in the Examples of WO2016/034849 are lithium salts of nucleotides, since these are marketed as the superior choice to those skilled in the art. Nucleotides supplied as a salt only of divalent cations, such as magnesium ions, are highly desirable, since the same are required during enzymatic DNA synthesis as a co-factor. Unfortunately, nucleotides provided as magnesium salts are highly insoluble and this limits their use. Instead, magnesium is usually supplied to the reaction separately as a chloride salt, in combination with nucleotides that are counter-ioned with a monovalent cation species.

The present inventors have previously found that the species of cation present in the nucleotide salt as the counter-ion is critical to the yield, efficiency and fidelity of high yielding enzymatic DNA synthesis reaction, as detailed in WO2020/035698 and WO2021/161051 (PCT/GB2021/050366), herein incorporated by reference.

The present inventors have developed a new family of novel nucleotide complexes, which may be powdered or may be in solution. The nucleotide complexes share an important feature, there is at least one divalent cation present at a ratio of 0.2-1.5 divalent cations per nucleotide. At the very simplest, the novel nucleotide complexes merely comprise a divalent cation present at a ratio of 0.2-1.5 divalent cations per nucleotide and no other cations are present to counter-ion the complex. The skilled person would understand that ions potentially present in any solvent (such as hydrogen and hydronium ions present in water) are exempt from this definition of cations. To this simplest nucleotide complex may be added further entities, such as a zwitterionic molecule. Nucleotide complexes comprising a mixture of a divalent cation and a zwitterion have been demonstrated by the Inventors to have particular utility is making large yields of DNA very quickly.

The present inventors have now developed a way of increasing the maximum tolerable starting nucleotide concentration for a DNA synthesis reaction further still, by the use of a process to remove the polyatomic moiety from monovalent cations from a nucleotide complex which is effectively counter-ioned by a mixture of divalent and monovalent cations, or by starting the process by removing the monovalent entity from a nucleotide complex associated with solely monovalent cations and then combining a nucleotide complex associated with solely divalent cations The starting material for the process may effectively be a mixed counter-ion nucleotide complex as described in WO2021/161051 (PCT/GB2021/050366), wherein the monovalent cation is polyatomic and the polyatomic moiety is rendered volatile under suitable conditions such as a vacuum.

The starting material may also be a single counter-ion nucleotide complex as described in WO2020/035698, for example including 4 ammonium ions. With this starting material, the divalent-counter-ioned nucleotide complexes may be added after the monovalent entities have been removed. The monovalent cation is polyatomic and the polyatomic moiety may be rendered volatile under suitable conditions such as a vacuum.

Either method may further permit the addition of a zwitterionic molecule before, during or after removal of the polyatomic moiety from the monovalent cation.

Using the processes of the invention, the inventors have found it is possible to further remove “monovalent cations” from the nucleotide complex in a manner that allows the dried nucleotide complex to be re-suspended in a solvent without the use of a buffering agent. This was entirely unexpected, most notably as it is already known that nucleotide salts with divalent cations such as magnesium are poorly soluble. The net effect is to reduce the amount of monovalent cation present even further than described in WO2021/161051 (PCT/GB2021/050366). This is important, since it is postulated by the inventors that monovalent cations are inhibitory in large concentrations to the DNA synthesis. This inhibition by monovalent cations may happen regardless of the source of the cations (nucleotide, buffer component etc.). Moreover, by also providing a divalent cation as a counter-ion to the nucleotide, particularly in the case of magnesium, manganese or calcium, this can effectively also provide the co-factor required by the synthesis enzyme. Therefore, it is not necessary to provide further or additional divalent cation. Since magnesium or manganese are usually added to the reaction as a salt (including two negative charges on one or more anions), including them instead as a counter-ion for the nucleotide inherently reduces the amount of anion present. Such a reduction may be beneficial for downstream DNA processing enzymes, since at the end of the synthesis reaction it would allow for fewer steps to prepare the DNA for further processing.

Further, the Inventors have surprisingly shown in the Examples that adding further molecules, such as zwitterions, to the novel nucleotide complex of the invention provides further new nucleotide complexes which have altered properties for DNA synthesis. These complexes appear to be particularly beneficial for accelerating the speed of DNA synthesis, particularly at starting concentrations above 100 mM.

The data shown the Examples demonstrate that the novel nucleotide complexes outperform previously prepared nucleotide complexes in terms of yield and efficiency, particularly at far higher concentrations of nucleotide entity (notably above 60 mM and even above 100 mM). Notably, DNA is used as the template to manufacture large quantities of RNA, particularly mRNA, and producing DNA on a commercial scale is therefore critical to the mass-production of RNA vaccines, for example. Thus, the requirement for clean, efficient DNA manufacture on an industrial scale is currently growing exponentially. The DNA produced using the present invention may be used as a template for manufacturing SARS-CoV-2 mRNA vaccines and the like. The DNA may be used in the preparation of virus particles, such as lentivirus and adeno-associated virus.

### SUMMARY

The present invention relates to novel nucleotide complexes which all require the presence of a divalent cation present at a ratio of 0.2-1.5 divalent cations per nucleotide. Effectively no further cations are present or supplied to “counter-ion” the nucleotide.

These divalent cations are preferably divalent metal cations, optionally one or more of magnesium, manganese or calcium. The divalent cation can be a single ion (for example magnesium) or a mixture of ions (for example magnesium and calcium).

In the simplest nucleotide complex, the novel nucleotide complexes comprise a divalent cation present at a ratio of 0.2-1.5 divalent cations per nucleotide and substantially no other cations are present to counter-ion the complex.

The skilled person would understand that ions naturally present in any solvents (such as hydrogen and hydronium ions present in water) are exempt from this definition of cations.

To this simplest nucleotide complex may be added further entities, such as a zwitterionic molecule. Nucleotide complexes comprising a mixture of a divalent cation and a zwitterion have been demonstrated by the Inventors to have particular utility is making large yields of DNA very quickly.

The present invention relates to a nucleotide complex comprising a nucleotide associated with a divalent cation present at a ratio of 0.2-1.5 divalent cations per nucleotide and a zwitterionic molecule present at a ratio of 0-4 zwitterionic molecules per nucleotide. Thus, the presence of the zwitterionic molecule is optional.

The present invention relates to a nucleotide complex comprising a nucleotide associated with a divalent cation present at a ratio of 0.2-1.5 divalent cations per nucleotide, and optionally further associated with a zwitterionic molecule which is present in an amount of less than or equal to 4 zwitterionic molecules per nucleotide.

The present invention relates to at least one process for the preparation of a novel nucleotide complex, wherein previously described nucleotide complexes (such as those in WO2021/161051) are dried, effectively to further reduce the volatile base of the monovalent cation included in the starting material. Such a process occurs when the monovalent cation chosen for the starting material is polyatomic and rendered volatile under suitable conditions such as vacuum. The nucleotide complexes after the drying or evaporation step may be partially or completely dried, for example most or all of the solvent may have been removed. Thus, it will be appreciated that the starting nucleotide complex may be in solution. The starting material for this process may simply be a mixture of nucleotide complexes associated with divalent cations together with nucleotide complexes associated with polyatomic monovalent cations.

The present invention permits the mixing of the nucleotides associated with said divalent cation and the nucleotides associated with said polyatomic monovalent cations at any appropriate point; this may be before the polyatomic moiety is removed, during removal or after removal. The removal of the polyatomic moiety is carried out by rendering it volatile under suitable conditions.

The processes of the invention may be considered as “drying” or “evaporation”, particularly if the starting nucleotide complex or the second nucleotide complex are in solution.

The drying process and subsequent re-suspension may allow for enhanced production of DNA compared to current methodologies, i.e. an increased or greater yield, a more efficient process or the ability to perform enzymatic DNA synthesis in an environment with fewer additional components than thought possible under current methodologies. This significantly increases productivity whilst reducing the cost of synthesising DNA, particularly on a large scale. In particular, such nucleotide complexes are shown to be suitable for use at particularly high starting concentration of nucleotides, such as 60 mM or even 100 mM and above.

In order to achieve a high yield at an industrial scale it is necessary to utilise high concentrations of the “building blocks” of DNA, the nucleotides (notably dNTPs). In general, the inventors have found that changing the parameters of the reaction conditions alone will not greatly increase the yields from an enzymatic reaction to make it suitable for industrial purposes.

Given the provision of nucleotides to the enzymatic reaction is as a salt, increasing the quantity of nucleotides results in a significant increase in the ionic strength of the reaction mixture. The ionic strength is a function of the concentration of all ions present. Such is an important consideration as the enzymes catalysing the DNA synthesis reaction are proteins, and an increased ionic strength can result in protein unfolding and thus inactivation of enzyme activity.

Further, it can be considered that the presence of a salt can also influence the pH of the reaction mixture. Depending on the acid-base properties of the component ions, a salt can dissolve in water to provide a neutral solution (strong acid/strong base), a basic solution (weak acid/strong base) or acidic solution (strong acid/weak base). Thus, by increasing the concentration of a nucleotide salt, or any other salt to the reaction mixture (magnesium chloride as an example), this may also influence the pH and further limit the pH stabilising performance of any buffering agent present. Thus, the addition of a higher concentration of nucleotide salt, for example, may lead to sub-optimal pH control, which impacts on the enzymatic DNA synthesis notably in terms of lowering DNA yields or adversely affecting the fidelity of DNA synthesis. Therefore, there are numerous considerations to be made in relation to the “scaling-up” of enzymatic DNA manufacture, and the present inventors have devised ways to increase the yield without adversely affecting the DNA synthesis reaction.

Without wishing to be bound by theory, the inventors postulate that phosphate groups on the nucleotide complex may provide some buffering capacity in solution. Further, as the DNA synthesis reaction proceeds, the reaction may self-buffer due to the release of phosphate from the nucleotide complexes. Measurements taken show that although the pH reduces after the nucleotide complex has been prepared, it did not reduce as far as expected, given the likely protonation of the nucleotide once the volatile moiety has been removed.

The present invention therefore relates to a process for improving the previously described nucleotide complexes further, by subjecting the same to a drying process, optionally to a point where no solvent remains. The starting material for the preparation of the nucleotide complex of the present invention may be a mixed counter-ion nucleotide complex, such as those described in WO2021/161051 (PCT/GB2021/050366). Such may include the presence of a divalent cation and a polyatomic monovalent cation that is rendered volatile under suitable conditions, such as a vacuum. The starting material may be a single counter-ion nucleotide complex or salt as described in WO2020/035698.

The present invention relates to a process for the preparation of a nucleotide complex comprising the step of applying heat and/or a vacuum to a starting nucleotide complex, wherein said starting nucleotide complex comprises a nucleotide associated with a polyatomic monovalent cation, wherein the polyatomic moiety is rendered volatile under heat and/or vacuum.

The present invention related to a process for the preparation of a nucleotide complex comprising the step of evaporating a polyatomic moiety present in a starting nucleotide complex, wherein said starting nucleotide complex comprises a nucleotide associated with a polyatomic monovalent cation.

The starting nucleotide complex according to any aspect of the invention may also comprise a divalent cation. Alternatively a nucleotide complex comprising a nucleotide associated with a divalent cation may be added to the starting material before or after the process has been performed.

Alternatively, the polyatomic moiety can be described as “volatisable”.

Alternatively presented, the present invention provides:

A process for the preparation of a nucleotide complex comprising:
(i) providing a starting nucleotide complex which comprises a nucleotide associated with a polyatomic monovalent cation, wherein the polyatomic moiety of the cation is volatisable;
(ii) applying any one or more of a heat, a vacuum and/or change in pH, to the starting nucleotide complex to enable a proportion of the polyatomic moieties to vaporise; and
(iii) before, during or after step (ii) mixing the starting nucleotide complex with a second nucleotide complex, said second nucleotide complex comprises a nucleotide associated with a divalent cation.

The polyatomic moieties are volatisable. This means that they may be rendered volatile under suitable conditions, but are generally not volatile under normal/standard conditions. Suitable conditions may be the application of heat, a vacuum and/or alteration in pH of the nucleotide complex. The pH may be changed to increase or decrease the pH, preferably increasing the pH.

Said method may further comprise:
(iv) before, during or after step (ii) mixing the starting nucleotide mixture with a zwitterionic molecule.

The starting nucleotide complex may be a nucleotide associated with a polyatomic monovalent cation present at a ratio of between 0.2 and 4 polyatomic monovalent cations per nucleotide, optionally between 0.5 and 3, between 1 and 2, or any ratio in between. The cation may be a sole species of polyatomic monovalent cation, or it can be a mixture of different species.

The second nucleotide complex may be a nucleotide associated with a divalent cation present at a ratio of between 0.2 and 4 divalent cations per nucleotide, optionally between 0.5 and 3, between 1 and 2, or any ratio in between. The cation may be a sole species of divalent cation, or it can be a mixture of different species.

The present invention relates to a process for the preparation of a nucleotide complex by evaporating a polyatomic moiety present in a starting nucleotide complex, wherein said starting nucleotide complex is associated between 0.2 and 2 divalent cations and between 0.2 and 2.5 polyatomic monovalent ions per nucleotide.

The novel nucleotide complexes can be used as prepared according to any method of the invention. Alternatively, the novel nucleotide complexes may be provided with at least one zwitterionic molecule, prior to or after the process to evaporate the polyatomic moieties. Further, the present invention relates to enzymatic DNA synthesis using a nucleotidyltransferase and any of the nucleotide complexes prepared according to the present invention, such as a polymerase enzyme or other DNA synthesising enzyme, any of which can optionally be engineered to give it particular properties.

The present invention may relate to DNA synthesis from a nucleic acid template or DNA synthesis de novo, without a template, depending on the nucleotidyltransferase used.

The present invention may relate to isothermal methods of synthesising DNA that do not require temperature to be cycled via heating and cooling during amplification, but may allow for the use of heat to initially denature the template, if present. The invention preferably relates to the use of polymerase enzymes that are capable of replicating a nucleic acid template via strand-displacement replication, independently or with the help of other enzymes.

The processes of the invention involve the use of a starting material (starting nucleotide complex) and optionally a second nucleotide complex, that are nucleotides in the forms of complexes with associated ions, which may also be referred to herein as counter-ions. The nucleotide complex is generally present in solution, and therefore the associated counter-ion may or may not be dispersed in solution. Due to the nature of their preparation, the counter-ions may effectively be “shared” between the nucleotides, such that there is a ratio of counter-ion to nucleotide which is not a whole number. In solution with the nucleotide, ionic species are divalent counter-ions and polyatomic monovalent counter-ions, such that the partial or complete charge balance for each nucleotide is contributed by a mixture of monovalent and divalent cations. The provision can be such that the complex is electrically neutral, or may have a net negative charge. The starting nucleotide complexes may be supplied in solution, or may be dispersed in solution by adding solid nucleotide complexes to solution.

Alternatively, the starting complexes may be a nucleotide associated with only monovalent cations, which are mixed during the process with a nucleotide associated with only divalent cations. Due to the nature of their preparation, the counter-ions may effectively be “shared” between the nucleotides, such that there is a ratio of counter-ion to nucleotide which is not a whole number.

The starting nucleotide complexes are in solution, and comprise a nucleotide (also described here as a nucleotide ion or ionic species) associated with at least two different positive counter-ions (cations). It is preferred that one of these counter-ions is a polyatomic monovalent cation, i.e. it has a single positive charge due to the loss of one electron. The polyatomic moiety of the monovalent cation should be capable of becoming volatile under suitable conditions, such as under a vacuum. It is volatisable or volatizable. It will be understood that at normal temperature and pressure (NTP is 20° C. and 1 atm) that the polyatomic moiety of the polyatomic monovalent cation is preferably not substantially volatile and that suitable conditions are applied to render the moiety volatile. It is preferred that one of these counter-ions in the starting nucleotide complex is a divalent cation, i.e. it has a double positive charge due to the loss of two electrons. For the avoidance of doubt, these divalent counterions are not lost, removed or evaporated during the process of the invention. Alternatively, the divalent counterions may be provided during the process by addition in the form of a nucleotide complex associated with the divalent cations.

The starting nucleotides may be provided as a complex with a mixed counter-ion provision of polyatomic monovalent and divalent cations. The starting nucleotides may be provided as a complex with a counter-ion provision of a polyatomic monovalent cation.

It will be understood that in order to be processed using the invention, the polyatomic monovalent cation will be capable of being rendered volatile under suitable conditions such as a vacuum. Simple metallic monovalent cations, such as lithium, sodium and potassium are therefore not suitable for use as a counter-ion in the starting material, since these are neither polyatomic nor can they be rendered volatile under suitable conditions such as a vacuum. A very small proportion of the monovalent counterions present in the nucleotide complex may be such metal ions, due to contaminants in the original manufacturing process.

Thus, the starting nucleotide complex may consist essentially of a nucleotide and associated monovalent polyatomic cation(s) and optionally divalent cation(s). Some small amount of monovalent metal ion contamination may be present, but at concentrations that are immaterial to the invention. An immaterial amount may be a concentration less than 5%, less than 4%, less than 3%, less than 2% or less than 1% of the concentration of the nucleotide complex. In some embodiments, the starting nucleotide complex is in solution and additional entities may be added, to assist with evaporating or volatising the polyatomic moiety, or to provide additional stability to the novel nucleotide complex formed. Such entities do not generally include metal ions, but are instead polyatomic and preferably include zwitterionic molecules. Such entities are preferably not volatisable under the same conditions as the polyatomic moiety.

It will be understood that as a nucleotide has four negative charges, it is usual to provide four positive charges, generally through 4 monovalent cations to maintain electrical neutrality, and most commercial nucleotide salts are obtained on this basis. As a starting material, when the nucleotide complex is in solution, it is possible that less than 4 positive charges are supplied by the polyatomic monovalent and/or divalent cations used as the counter-ions. Without wishing to be bound by theory, the inventors postulate that the remaining charges may be provided by entities such as the hydronium ion, in order to reach electrical neutrality, if required. If additional entities such as zwitterions are present, these are thought by the inventors to help to stabilise the complex. Once the nucleotide complex has been subjected to the process of the invention, a proportion or substantially all of the polyatomic moiety is removed or evaporated. A proportion may be 50%, 55%, 60%, 70%, 75%, 80%, 85%, 90%, 95%, 96%, 97%, 98%, or 99% removed or evaporated (when compared to the starting nucleotide complex). Preferably 100% of the moiety is removed or evaporated. It is preferred that less than 0.2 polyatomic monovalent cation per nucleotide remains, optionally less than 0.1, preferably none.

The polyatomic moiety of the polyatomic monovalent cation is volatisable and may be rendered volatile under any suitable condition. The polyatomic moiety is preferably not rendered volatile under normal temperature and pressure. The polyatomic moiety is herein described as being rendered volatile. Alternative ways of describing such a moiety are: evaporable, vapourific, vaporisable or volatilisable. Volatility is indicated by a substance's vapour pressure. Substances with a higher vapour pressure will vaporise more readily under suitable conditions than a substance with a lower vapour pressure. At a given temperature and pressure, a substance with high vapour pressure is more likely to exist as a vapour, while a substance with low vapour pressure is more likely to be a liquid or solid. Suitable conditions are selected to vaporise the substance.

Suitable conditions under which to render to moiety volatile are temperature (for example the application of heat) or pressure (for example the application of a vacuum). Some polyatomic entities may be rendered volatile by a change in pH, such as increasing the pH (for example by including a basic amino acid). The most suitable conditions under which to render the polyatomic moiety volatile are heat and/or vacuum.

The polyatomic monovalent cation may be rendered volatile under a vacuum. The temperature at which the cation is rendered volatile under a vacuum may any suitable temperature, such that the complex in solution may be at room temperature (approximately 20° C.) or may be cooled or heated. As used in the Examples, heat may be applied to assist with rendering the polyatomic monovalent cation volatile. Heat may be applied in the range of 20-80° C., 25-75° C., 30-70° C., 35-65° C. or at any suitable temperature therebetween, such as 20, 25, 30, 35, 40, 45, 50, 55, 60, 65, 70, 75 or 80° C.

A vacuum may create a region with a gaseous pressure less than atmospheric pressure. Thus, the gaseous pressure may be any appropriate pressure ranging from 10⁵ Pascal (Pa) to less than 10⁻¹⁰ Pa. Ideally, the vacuum will be created at a pressure of 10⁵ Pascal (Pa) to 100 Pa. Standard atmospheric pressure is 101,325 Pa. A vacuum may be created using any appropriate technique, including the use of a vacuum pump or by any other method of reducing the pressure, such as the use of fast-flowing liquids.

The polyatomic monovalent cation may be rendered volatile under heat. Heat may be applied in the range of 40-100° C., 45-95° C., 50-90° C., 55-85° C. or at any suitable temperature therebetween, such as 40, 45, 50, 55, 60, 65, 70, 75, 80, 85, 90, 95 or 100° C.

The monovalent ion as described herein is polyatomic, such that it is an ion that consists of more than one atom. One of those atoms may be nitrogen, for example a protonated nitrogen atom. An exemplary ion is the ammonium ion (NH₄⁺).

The polyatomic monovalent cation may be described as the conjugate acid of a base, wherein the base is a volatile moiety. For example in relation to ammonium under a vacuum:

$${H}_{2} O ( I ) + {NH}_{3} ( g ) ⇋ {NH}_{4}^{+} + {OH}^{-} ( aq )$$

$$Acid Base * Conjugate Acid * Conjugate Base$$

In this Example, the ammonium ion is the conjugate acid of ammonia (both marked *).

Thus, the polyatomic monovalent cation may be described as a conjugate acid of a base, wherein the base is a volatile moiety. In general, the base is the species formed after the conjugate acid donates a proton. The monovalent cation may alternatively be described as a Brønsted-Lowry acid, wherein the conjugate base is volatile. It is hypothesized that the proton is donated to the nucleotide entity in the process of the present invention. Thus, the moiety that is evaporated or removed in the process is the base—referred to herein as the “polyatomic moiety”.

Exemplary conjugate bases and acids include ethylamine base and ethylammonium conjugate acid, methylamine base and methylammonium conjugate acid. Both are derivatives of ammonium and both lose a proton to form a volatile conjugate base (polyatomic moiety).

The polyatomic monovalent cation may therefore comprise a nitrogen atom, preferably a protonated nitrogen atom.

The polyatomic monovalent cation may be ammonium or a derivative thereof. Derivatives of ammonium are also encompassed, and a non-limiting, exemplary list of these includes: monoalkyl ammonium or dialkyl ammonium. Those skilled in the art will be aware of derivatives of ammonium that carry a single positive charge that are appropriate to use in the present invention.

Alternatively, the polyatomic monovalent cation may be described as an organic cation. Usually, ammonium is described as an inorganic ion, but many of the derivatives are organic, and the ammonium ion may be grouped together with its derivatives thereof, which are organic. Organic nitrogen occurs in many forms, including in organic solvents, peptides and proteins.

Accordingly there is provided a process of preparing a novel nucleotide complex, comprising drying a nucleotide complex, wherein said complex comprises a nucleotide and a divalent cation present at a ratio of between 0.2 and 2 divalent cations and a polyatomic monovalent cation present at a ratio of between 0.2 and 2.5 polyatomic monovalent cations per nucleotide. The drying is preferably performed under heat and/or vacuum, and the polyatomic moiety of the monovalent cation is rendered volatile under these conditions. Preferably there are between 0.2 and 1 divalent cations present per nucleotide.

Accordingly there is also provided a process of preparing a novel nucleotide complex, comprising removing a polyatomic moiety from a starting nucleotide complex, wherein said starting nucleotide complex comprises a nucleotide and a polyatomic monovalent cation present at a ratio of between 0.2 and 4 polyatomic monovalent cations per nucleotide. The starting nucleotide complexes may be mixed with a second nucleotide complex, said second nucleotide complex comprises a nucleotide and a divalent cation present at a ratio of between 0.4 and 2 divalent cations per nucleotide. This mixing step may be before or after the removal of the polyatomic moiety.

Accordingly there is also provided a process of preparing a novel nucleotide complex, comprising removing a polyatomic moiety from a starting nucleotide complex, wherein said starting complex comprises a nucleotide and a divalent cation present at a ratio of between 0.2 and 2 divalent cations and a polyatomic monovalent cation present at a ratio of between 0.2 and 2.5 polyatomic monovalent cations per nucleotide.

The removal is preferably performed under altered pH, heat and/or vacuum, and the polyatomic moiety of the monovalent cation is rendered volatile under these conditions.

According to any process of the invention, the drying, evaporation or removal of the volatile moiety is preferably conducted under a vacuum.

Those skilled in the art would appreciate that the nucleotide complex used as the starting material will generally be in solution to enable it to be dried or for the volatisable moiety to evaporate or be removed.

It will be appreciated that the term nucleotide as used here may also be read as a nucleotide ion or nucleotide ionic species; it is the nucleotide entity without any counter-ion present.

Further, there is provided:

A cell-free process for the enzymatic synthesis of DNA comprising obtaining a nucleotide complex in solution, wherein said complex is prepared according to any process or method described herein, and adding a nucleotidyltransferase.

Once prepared it is thought that the nucleotide complexes of the invention comprise substantially none of the originally associated monovalent cations. Thus, it is expected that the level has decreased to between zero and 1 monovalent cation present per nucleotide, preferably between 0 and 0.5 per nucleotide, optionally less than 0.2 monovalent cations per nucleotide, such as 0.2. 0.1 or zero monovalent cations per nucleotide. As such, a proton or hydronium ion is not considered a monovalent cation for the purposes of the invention.

The present invention therefore provides a novel nucleotide complex. The novel nucleotide complex is a nucleotide associated with a divalent cation present at a ratio of between 0.5 and 1.5 divalent cations and a polyatomic monovalent cation present at a ratio of less than 0.5 monovalent cations per nucleotide. Such a novel nucleotide complex may be present in a solvent, such as water. The nucleotide complex in solution may include substantially no further cations, other than residual metal monovalent cations which may be a manufacturing contaminant from the nucleotides themselves. Suitable tolerable levels of these are disclosed herein.

The present invention therefore provides a novel nucleotide complex solution consisting effectively of: a nucleotide, a divalent cation present at a ratio of between 0.5 and 1.5 divalent cations per nucleotide and a monovalent cation present at a ratio of less than 0.5 monovalent cations per nucleotide.

Preferred are a novel nucleotide complex solution wherein a/the divalent cations are present at a ratio of between 0.5 and 1.5, 0.6 to 1.4, 0.7 to 1.3, 0.8 to 1.2, 0.9 and 1.1 or simply one divalent cations present per nucleotide.

Preferred are a novel nucleotide complex where a/the monovalent cations are present at a ratio of less than 0.5, 0.4, 0.3, 0.2, 0.1 or indeed no monovalent cations are present per nucleotide.

Any combination of the above is feasible using the process of the invention.

The divalent cations are preferably calcium, magnesium or manganese.

The inventors postulate that where the “charge” of the associated divalent cation does not balance the charge on the nucleotide, that hydronium ions (H₃O*) provide additional positive charges. Thus, for all of the nucleotide complexes described herein, hydronium ions may be present.

These ions may be present at a ratio of 0.5 to 3.5, optionally 1 to 3, optional 1.5 to 2.5, optionally around 2 ions per nucleotide. These are not actively added, they are formed when the nucleotide complex is prepared according to the processes of the invention.

The process of the invention may further comprise adding to the starting nucleotide complex or the novel nucleotide complex one or more zwitterionic molecules. Such zwitterionic molecules are preferably not rendered volatile under the conditions used in the processes of the invention.

Zwitterions contain both positive- and negative-charged groups with a net charge of zero. Some zwitterions cannot isomerise away from containing positive- and negative-charged groups, however most are able to lose all charge and become completely neutral, this is often at neutral pH (pH7) but not exclusively so. At different pH the zwitterionic molecules may gain or lose hydronium ions in solution, rendering them charged and converting the molecule away from the zwitterion form. The zwitterionic molecule may be an amino acid, where the negative charge comes from the carboxylate ion while the positive charge comes from the ammonium ion. The zwitterionic molecule may be an ylide. Ylides are neutral dipolar molecules containing a formally negatively charged atom (usually a carbanion) directly attached to a heteroatom with a formal positive charge (usually nitrogen, phosphorus or sulphur). Ylides is a sub-class of zwitterions which are polar; in which the atom with positive and the atom with negative charge are bonded together i.e. X⁺—Y⁻. Both an amino acid and an ylide are added to the nucleotide complexes exemplified herein.

In the case of amino acids in particular, any suitable enantiomer may be selected. Exemplified herein is the L-enantiomer of histidine. L-enantiomers are generally seen biologically, and may therefore be preferred. Amino acids that are preferable are Lewis acid:bases. Zwitterions that are Lewis acid:bases may be suitable for the invention.

The maximum number of zwitterions that could be associated with a nucleotide complex are 4, assuming one lone positive charged group, but further zwitterions could be present in the solution.

The present invention therefore provides a novel nucleotide complex. The novel nucleotide complex is a nucleotide associated with a zwitterionic molecule which is present in an amount of less than or equal to 4 zwitterionic molecules per nucleotide, a divalent cation present at a ratio of between 0.5 and 1.5 divalent cations per nucleotide and optionally a monovalent ion present at less than 0.5 monovalent cations per nucleotide. Such a novel nucleotide complex may be present in a solvent, such as water. The nucleotide complex in solution may include substantially no further or additional cations or ions, other than residual metal monovalent cations which may be a manufacturing contaminant from the nucleotides themselves.

The present invention therefore provides a novel nucleotide complex solution consisting effectively of: a nucleotide, zwitterionic molecule present in an amount of less than or equal to 4 zwitterionic molecules per nucleotide, divalent cations present in an amount of between 0.5 and 1.5 divalent cations per nucleotide and optionally monovalent cations present in an amount of less than 0.5 monovalent cations per nucleotide.

Preferred are a novel nucleotide complex solution wherein the a/the divalent cation is present at a ratio of between 0.5 and 1.5, 0.6 to 1.4, 0.7 to 1.3, 0.8 to 1.2, 0.9 and 1.1 or simply one divalent cation is present per nucleotide.

Preferred are a novel nucleotide complex where a/the monovalent cation is present at a ratio of less than 0.5, 0.4, 0.3, 0.2, 0.1 or indeed no monovalent cations are present per nucleotide.

Preferred are a novel nucleotide complex where a zwitterion molecule is present at a ratio 0-4, 0-3, 0.5 to 2.5, 1 to 2, or 1.5 zwitterionic molecules present per nucleotide. AS used herein zwitterion and zwitterionic are used interchangeably.

Any combination of the above is feasible using the process of the invention.

The divalent cations are preferably calcium, magnesium or manganese, or a mixture thereof.

The zwitterion molecule is preferably an amino acid or an ylide, and preferred amino acids are histidine, lysine and arginine.

Provided is a novel nucleotide complex which comprises a nucleotide associated with a divalent cation present at a ratio of between 0.5 and 1.5 divalent metal cations per nucleotide, and a zwitterion molecule present at a ratio of between 0.5 and 4 zwitterion molecules per nucleotide.

Preferred is a novel nucleotide complex which comprises a nucleotide associated with between 0.5 and 1.5 calcium, magnesium and/or manganese per nucleotide, and between 0.5 and 4 molecules selected from any one or more of histidine, lysine, arginine or dimethylsulfoxonium-(isobutanoyl)methylide.

It will be appreciated that any of the above ratios can be applied to these novel nucleotide complexes. The novel nucleotide complex may also consist essentially of the listed entities, plus a solvent if required. Other ions are preferably absent, or at very low levels as discussed previously (less than 5%, 4%, 3%, 2% or 1% of the nucleotide concentration is contaminating ions).

The present invention further extends to a cell-free process for the enzymatic synthesis of DNA comprising the use of any of the novel nucleotide complexes described herein.

A cell-free process for the enzymatic synthesis of DNA comprising obtaining a nucleotide complex in solution, wherein said complex is a nucleotide associated with a divalent cation present at a ratio of between 0.2 and 1.5 divalent cations and monovalent cations present at a ratio between 0 and 1 monovalent cations per nucleotide, in combination with a nucleotidyltransferase. Optionally the nucleotide complex may also be associated with a zwitterion molecule present in an amount of less than or equal to 4 zwitterionic molecules per nucleotide

A cell-free process for the enzymatic synthesis of DNA using a nucleotidyltransferase comprising combining said enzyme and a nucleotide complex, wherein said complex is a nucleotide associated with a divalent cation present in a ration of between 0.2 and 1.5 divalent cations and a monovalent cation present in a ratio of between 0 and 1 monovalent cations per nucleotide. Optionally the nucleotide complex may also be associated with a zwitterion molecule present at a ratio between 0 and 4 zwitterion molecules per nucleotide.

Also provided is a novel nucleotide complex in solution comprising a nucleotide, a divalent cation present at a ratio between 0.2 and 1.5 divalent cations, and a monovalent cation present at between 0 and 1 monovalent cations per nucleotide. Optionally the nucleotide complex may also be associated with a zwitterion molecule present at between 0 and 4 zwitterion molecules per nucleotide.

Also provided is a novel nucleotide complex in solution consisting essentially of a nucleotide, between 0.2 and 1.5 divalent cations, and between 0 and 1 monovalent cations per nucleotide. Optionally the nucleotide complex may also be associated with between 0 and 4 zwitterion molecules per nucleotide.

It will be appreciated that the term nucleotide as used here may also be read as a nucleotide ion or nucleotide ionic species; it is the nucleotide entity without any counter-ion present.

It will be appreciated that in solution the ions forming the complex may or may not dissociate.

In relation to the nucleotide complex, reference to monovalent cations as used herein does not usually include protonation or association with a hydronium ion. Either may be present in the nucleotide complex in solution to balance the charges present.

It is preferred that the enzymatic DNA synthesis is for the manufacturing of DNA on a larger scale, i.e. for therapeutic or prophylactic use (stated as grams per litre reaction mixture), rather than lab-scale amplification (scale of ng to mg per litre). It is in this scaling-up of laboratory scale amplification that the present inventors have found that it is not as simple as providing more substrate and other components and finding that the yield follows suit. Thus, the process comprises the use of nucleotide complexes in general at a concentration equal or greater than 30 mM, said concentration being determined when the nucleotide complexes are combined with the nucleotidyltransferase. The mixing of the nucleotide complexes and the enzyme results in the formation of a reaction mixture. Since the nucleotide complex is dried (or the volatile polyatomic moiety is evaporated), this may simply be added to a solution containing the nucleotidyltransferase. The concentration is determined in the reaction mixture in which the process is performed. Thus, the concentration of the nucleotide complexes is determined in the reaction mixture at the time that the nucleotide complexes are added. The concentration is therefore the initial concentration or concentration at the initiation of the process.

Accordingly, there is provided a cell-free process for the enzymatic DNA synthesis comprising the use of nucleotide complexes at a concentration of at least 30 mM in solution, wherein said complexes are prepared according to any method described herein.

Optionally the complexes may be present at a concentration of at least 40 mM, 50 mM, 60 mM, 70 mM, 80 mM, 90 mM, 100 mM, 110 mM or 120 mM. Optionally the complexes are present at a concentration of 40 mM to 160 mM, 50 mM to 150 mM, 60 mM to 140 mM, 70 mM to 130 mM or 80 mM to 120 mM, or any range between these values.

Without wishing to be bound by theory, the nucleotide complexes prepared according to the methods described herein comprise a nucleotide associated with between 0.2 and 1.5 divalent cations and between 0 and 1 monovalent cations, wherein said monovalent cations are polyatomic. Optionally the nucleotide complex may also be associated with between 0 and 4 zwitterion molecules per nucleotide. All of these may be described as being present in the nucleotide complex.

According to any aspect of the invention, the nucleotide complexes are substantially free of any monovalent metal cations, such as lithium (concentration of less than 1% nucleotide concentration), sodium (less than 5%), and potassium (less than 5%). If a denaturant, such as sodium hydroxide, is required during the DNA synthesis, this may be added to the reaction mixture once the nucleotide complex has been prepared.

Accordingly, there is provided a cell-free process for the enzymatic synthesis of DNA comprising obtaining a nucleotide complex in solution at a concentration of at least 30 mM, said complex comprising a nucleotide associated with a divalent cation present at a ratio between 0.2 and 1.5 divalent cations and a polyatomic monovalent cation present at a ratio between 0 and 1 polyatomic monovalent cations per nucleotide, and adding a nucleotidyltransferase. Optionally the nucleotide complex may also be associated with a zwitterion molecule present at a ratio of between 0 and 4 zwitterion molecules per nucleotide. Optionally the complexes may be present at a concentration of at least 40 mM, 50 mM, 60 mM, 70 mM, 80 mM, 90 mM, 100 mM, 110 mM or 120 mM. Optionally the complexes are present at a concentration of 40 mM to 160 mM, 50 mM to 150 mM, 60 mM to 140 mM, 70 mM to 130 mM or 80 mM to 120 mM, or any range between these values.

The enzymatic DNA synthesis may involve any enzyme capable of synthesising DNA, notably a nucleotidyltransferase, the definition of which herein includes all enzymes capable of transferring a nucleotide to the end of a nascent polynucleotide chain, either on the basis of a template or de novo. The nucleotidyltransferase may include a polymerase or a modified polymerase, such as a DNA polymerase or an RNA polymerase. The polymerase may be from any of the known families of DNA polymerase, including families A, B, C, D, X, Y and RT. An example of a DNA polymerase from family X is terminal deoxynucleotidyl transferase.

The nucleotidyltransferase may be present in solution, and the nucleotide complexes can be added as a solid preparation to the enzyme in solution, for example as a lyophilised powder.

The enzymatic DNA synthesis may occur de novo without the use of a template.

The enzymatic DNA synthesis may involve a template, such as a nucleic acid template, including a DNA template.

The enzymatic DNA synthesis may take place in a reaction mixture, comprising the components described here.

Alternatively written, there is provided a cell-free process for synthesising DNA in solution comprising contacting a template with at least one nucleotidyltransferase in the presence of one or more nucleotide complexes, wherein said nucleotide complexes comprise a nucleotide associated with a divalent cation present at a ratio between 0.2 and 1.5 divalent cations and a polyatomic monovalent cation present at a ratio between 0 and 1 polyatomic monovalent cations per nucleotide. Optionally the nucleotide complex may also be associated with a zwitterion molecule present at a ratio of between 0 and 4 zwitterion molecules per nucleotide. Optionally the concentration of said nucleotide complexes is at least 30 mM, preferably 40 mM. Optionally the complexes may be present at a concentration of at least 40 mM, 50 mM, 60 mM, 70 mM, 80 mM, 90 mM, 100 mM, 110 mM or 120 mM. Optionally the complexes are present at a concentration of 40 mM to 160 mM, 50 mM to 150 mM, 60 mM to 140 mM, 70 mM to 130 mM or 80 mM to 120 mM, or any range between these values.

Alternatively put, the nucleotide complexes include a mixture of divalent and polyatomic monovalent cations, along with the nucleotide itself. Optionally the nucleotide complex may also be associated with a zwitterion molecule present at a ratio of between 0 and 4 zwitterion molecules per nucleotide. Accordingly, there is provided a cell-free process for synthesising DNA comprising contacting a template with at least one nucleotidyltransferase in the presence of one or more nucleotide complexes to form a reaction mixture, wherein said nucleotide complexes are present at a concentration of at least 30 mM and comprise a nucleotide associated with a divalent cation present at a ratio between 0.2 and 1.5 divalent cations and a monovalent cation present at a ratio between 0 and 1 monovalent cations. Optionally the nucleotide complex may also be associated with a zwitterion molecule present at a ratio of between 0 and 4 zwitterion molecules per nucleotide.

It is preferred that when the concentration of the nucleotide or nucleotide complex is referred to, this is the concentration of nucleotide (or complex thereof) when the DNA synthesis process begins, i.e. the starting or initial concentration of nucleotide (or nucleotide complex). Thus, it is the concentration after addition to the reaction mixture. It will be appreciated that addition of other components can be made during the process; such additions may dilute the concentration of the nucleotides/nucleotide complexes, unless further nucleotides/nucleotide complexes are supplied to replenish the concentration. Further, since the nucleotide/nucleotide complexes will be used or consumed by the process, i.e. the DNA synthesis reaction, the concentration of the nucleotide/nucleotide complexes will fall as the process progresses. In certain embodiments, further nucleotides/nucleotide complexes may be added as the process progresses in order to replenish substrates for the enzymatic reaction. Ideally, the concentration of the nucleotides in the DNA synthesis reaction will be kept in the range of 40 mM to 160 mM, 50 mM to 150 mM, 60 mM to 140 mM, 70 mM to 130 mM or 80 mM to 120 mM, or any range or value between these values. The constant maintenance of nucleotide concentration can be beneficial to the DNA synthesis reaction.

The inventors have surprisingly found that if nucleotide complexes which include a mixture of polyatomic monovalent and divalent cations are dried under heat and/or a vacuum and subsequently re-suspended, that the generated nucleotide complex can be used in a DNA synthesis reaction with a polymerase, wherein the efficiency of conversion of the nucleotides into a DNA remains constant at varying concentrations. The Examples note that the efficiency of the reaction using these novel nucleotides remains in the order of 55-65% regardless of concentration tested between 40 mM and 120 mM. Such an effect has not hitherto been observed by the present inventors, who have previously noted that other complexes have a particular “peak” at which they prefer to operate. This further improvement is compared to a conventional nucleotide salt with 4 monovalent cations or a mixed nucleotide complex as previously described. Gaining the same efficiency of conversion of the nucleotide complexes at 120 mM is particularly exciting, since it offers the ability to scale up without loss of efficiency.

The inventors have further demonstrated in the Examples that, particularly at concentrations above 60 mM, the novel nucleotide complexes of the invention, particularly those including one magnesium ion (without or without a zwitterionic molecule) outperforms a nucleotide ammonium salt (4 ammonium ions). Greater yields of DNA are generated.

The addition of a zwitterionic molecule to the nucleotide complex of the invention has been observed to accelerate the production of DNA, such that a higher yield is achieved more rapidly, particularly at concentrations above 100 mM nucleotide complex. This was observed for both zwitterionic molecules tested. Indeed, 16 g/l DNA was synthesised in only 48 hours according to Example 7.

The present invention therefore provides a cell-free process for the enzymatic manufacture of at least 15 g/L DNA in a period of between 40 and 55 hours, comprising the use of a nucleotidyltransferase and a nucleotide complex comprising a nucleotide associated with about 1 magnesium or manganese ion and at least one zwitterionic molecule, optionally histidine, lysine, arginine or dimethylsulfoxonium-(isobutanoyl)methylide.

All components of this nucleotide complex are as previously described.

The inventors have also noted that the association of magnesium or manganese cations with the nucleotide complex means that no additional magnesium or manganese is required in the reaction mixture in order to synthesize DNA. This is beneficial in reducing components and therefore cost. Further, it means that the ionic strength of the reaction mixture may be further reduced compared to the prior art, which the inventors have found to be favourable for DNA synthesis reactions. Such conditions may be ideally suited to various synthesis reactions, such as the production of single stranded DNA. The inventors have noted in the work conducted to date that the nucleotide complexes as described here for the first time are appearing to produce more single stranded DNA than the parent nucleotide complexes from which they are produced.

Convention dictates, for example, that magnesium (a divalent cation) is present in DNA synthesis reactions at a minimum ratio of at least 1:1 with the nucleotides. This is because magnesium may be required at the active site of some nucleotidyltransferase enzymes; it may form a complex with the nucleotide prior to incorporation and further may form its own salt with the phosphate ionic species released during DNA synthesis. The benefit of including magnesium or manganese in the nucleotide complex is that this reduces or removes the requirement for additional magnesium or manganese. It assists to maintain an approximate 1:1 ratio (or below 1:1 ratio) of magnesium or manganese to nucleotide, which the inventors have previously identified as desirable in large scale DNA synthesis reactions. This is important, since reducing the components included in the DNA synthesis notably reduces costs, but furthermore a higher concentration (greater than 1:1) of magnesium is related to a decreased fidelity in DNA synthesis. Some DNA synthesis enzymes utilise calcium instead of magnesium, these include DP04. For these enzymes, including calcium as the divalent ion is attractive.

The divalent cations associated with the nucleotide in the complex may comprise one or more metals selected from the list consisting of: magnesium (Mg²⁺), beryllium (Be²⁺), calcium (Ca²⁺), strontium (Sr²⁺), manganese (Mn²⁺) or zinc (Zn²⁺), preferably Mg²⁺ or Mn²⁺. The ratio between the divalent metal cations and the nucleotide (nucleotide ion or nucleotide ionic species) may be about 1:1 in solution, but is preferably between 0.2:1 and 2:1, optionally 0.5:1 to 1.5:1. Ratios lower than 1:1 are desirable and are preferable in DNA synthesis since ratios higher than 1:1 may lead to some infidelity in DNA synthesis. The provision of the divalent cations in relation to the nucleotide complex may therefore reduce or remove the need to add additional divalent cations to the reaction mixture. However, should further be required, these divalent cations may be provided to the enzymatic DNA synthesis in the form of any suitable salt.

Further, the method developed here by the present inventors can be performed in a large range of conditions with respect to the other components present. These conditions range from a conventional level of buffering to the provision of no further buffering agents, effectively performing the reaction with the required components in water. Increasing the concentration of a buffering agent may increase buffering capacity to directly improve pH control but chemical buffers can also chelate a range of metal ions including magnesium ions and may adversely interfere with the balance of mono- and divalent cations required for optimal DNA yields. Using concentrations of buffering agents at the lowest possible levels while balancing other essential reaction components to maintain pH within an acceptable band for optimal DNA production may, therefore, be desirable. Those skilled in the art will appreciate that some of the counter-ions proposed here may have their own buffering ability, or entities released or produced during DNA synthesis (for example pyrophosphates and phosphates) may also help buffer against excessive pH change.

The required components, regardless of buffering agent provision, may include the DNA synthesising enzyme (nucleotidyltransferase), such as a polymerase, the nucleotide complexes, with optional additional components required depending on the conditions of the reaction, selected from divalent metal cations provided as a salt, a template, a denaturing agent, a pyrophosphatase, or one or more primers/primase. These components may form the reaction mixture. Thus, in its most basic form, the reaction mixture is simply the prepared nucleotide complexes plus a nucleotidyltransferase. It will be understood that it is desirable that the reaction mixture does not contain superfluous ionic species, since such entities may undesirably affect the DNA synthesis. Other than the ions present in the nucleotide complex, other ions may be present in minimal amounts, such as in denaturing agents (for example sodium or potassium hydroxide) or in buffering agents. It may be necessary to further supplement magnesium or manganese salts, depending on the nucleotide complex selected and enzyme involved in the reaction. It is preferred that the concentration of “additional ions” in the reaction mixture, such as at the start of the reaction, may be kept to a minimal level, for example less than 50 mM, less than 40 mM, less than 30 mM, less than 20 mM, or less than 10 mM. Such additional ions are ions other than those supplied with the nucleotide complex or generated therefrom during the course of the reaction.

Thus, the provision to the process, i.e. the reaction mixture, of nucleotides as complexes with a reduced mixed counter ion provision is advantageous, since this surprisingly allows for improved DNA yield and/or an improved efficiency of conversion of the nucleotides into DNA. These improvements may be compared to an analogous reaction mixture where all the nucleotides are supplied as conventional salts with the requisite monovalent cation alone. The provision of novel nucleotide complexes instead of nucleotide salts conventionally used has some further surprising advantages, such as the ability to lower the concentration of buffering agents in the reaction mixture, in some instances to zero, and/or to lower, decrease or remove entirely the requirement for the additional provision of a divalent cation co-factor for the reaction mixture, most notably magnesium, which is usually added as a salt. Given that this salt may no longer be required, the present invention has the effect of reducing the ionic strength of the reaction mixture, since if the divalent cation salt is not added, there is no provision to the reaction mixture of the associated anion (e.g. MgCl₂—therefore the addition of two chloride ions is avoided). The ionic strength of a solution is a measure of the concentration of ions in that solution. Ionic compounds, when dissolved in water, dissociate into ions. As used herein, the unit of measurement is molar (mol/L). The inventors postulate that a reduction in the ionic strength of the reaction mixture may be beneficial to the process. It is possible that the monovalent cations provided with the nucleotide entity are inhibitory to the process in high concentrations, and therefore by further removing them according to the processes of the invention, yet further improvements are possible. Alternatively or additionally, in a standard process, magnesium or manganese is supplied as a salt, and high concentrations of the associated anion from this salt may also be inhibitory to the process, for example the chloride ion. Furthermore, should it be desirable to manipulate the DNA produced using the invention, the inventors have seen that enzymes introduced into the reaction mixture, such as enzymes that cleave and ligate target sequences (DNA processing enzymes), prefer the conditions of lower ionic strength, since these are generally added at the end of the DNA synthesis reaction, when the ionic strength may have increased significantly with conventional nucleotide and divalent salts. Thus, the product from such a DNA synthesis reaction may be suitable for further processing enzymatically (such as use as a template to produce RNA using RNA polymerase).

In one aspect, a template directs the enzymatic DNA synthesis in the processes. This template may be any nucleic acid template, for example a DNA or RNA template. The template may be a natural nucleic acid, an artificial nucleic acid or a combination of the two. The amplification of the template is preferably via strand-displacement. The amplification of the template is preferably isothermal, i.e. there is no requirement to cycle between low and high temperatures to progress the amplification. In this scenario, heat may be used at the start to denature the template, if required, or the template may be denatured by chemical means. However, once the template has been denatured, if appropriate, to allow any primers or indeed primase enzyme to enter between double stranded templates, the temperature may be maintained at a range of temperatures that do not affect the denaturation of the template and product. Isothermal temperature conditions require that the reaction is not heated to a point where the template and products denature (compared to PCR which requires cycles of heat to denature the template and product). Generally such reactions are performed at a constant temperature, depending on the preference of the enzyme itself. The temperature may be any suitable temperature for the enzyme.

The cell-free process preferably involves amplification of the template via strand displacement replication. This synthesis releases a single stranded DNA, which may in turn be copied into double stranded-DNA, using a polymerase. The term strand displacement describes the ability to displace downstream DNA encountered during synthesis, wherein the polymerase opens the double-stranded DNA in order to extend the nascent single strand. DNA polymerases with varying degrees of strand displacement activity are available commercially. Alternatively, strand displacement can be achieved by supplying a DNA polymerase and a separate helicase. Replicative helicases may open the duplex DNA and facilitate the advancement of the leading-strand polymerase.

Independently, optional features of any aspect of the invention may be: The template may be circular. The DNA may be synthesised by amplification of a template, optionally by strand displacement replication. The strand displacement amplification of said DNA template may be carried out by rolling circle amplification (RCA). The polymerase may be Phi29, or a variant thereof. The amplification of DNA may be isothermal amplification, i.e. at a constant temperature. A primer or primase may be used to initiate amplification. A nickase may be used to generate a primer “in situ” on a double stranded circular template. The one or more primers may be random primers. A pair or set of primers may be used. The synthesised DNA may comprise concatamers comprising tandem units of DNA sequence amplified from the DNA template. The DNA template may be a closed linear DNA; preferably the DNA template is incubated under denaturing conditions to form a closed circular single stranded DNA.

The quantity of DNA that may be synthesised is equal to or higher than 3 g/litre of reaction mixture, notably 16 g/l or more, preferably up to 25 g/l and beyond.

The novel nucleotide complexes prepared according to the methods of the present disclosure may permit DNA synthesis at high starting concentrations, for example at 90 mM, 100 mM or even 120 mM and beyond.

The amount of DNA that may be synthesised may exceed 60% of the calculated maximum yield for the reaction mixture. Preferably, the amount of DNA that may be synthesised may exceed 80% of the calculated maximum yield. The calculated maximum yield is based upon the theoretical yield should all nucleotides be incorporated into a product, and this can be calculated by those skilled in the art.

The efficiency of DNA synthesis from the nucleotides (or nucleotide complexes) may be described as the percentage of nucleotides or complexes thereof supplied to the reaction mixture which are successfully incorporated into a product over the course of the reaction.

The efficiency of the DNA synthesis may also be maintained over a broader range of nucleotide concentrations by the present invention, resulting in an improved range of concentrations in which DNA synthesis can be performed.

The process for preparing novel nucleotide complexes requires at least one starting nucleotide complex. Any suitable number of phosphate groups may be present, as required. However, it is preferred that the nucleotides/nucleotide complexes are deoxyribonucleoside triphosphates (dNTPs) or a derivative or modified version thereof. The nucleotides are one or more of deoxyadenosine triphosphate (dATP), deoxyguanosine triphosphate (dGTP), deoxycytidine triphosphate (dCTP), deoxythymidine triphosphate (dTTP) and derivatives thereof. Each individual starting nucleotide complex may, but is not required to, be charged balanced with various cations providing 4 positive charges to maintain electrical neutrality. The starting nucleotide complexes used in the drying process may include one or more polyatomic monovalent cations, i.e. one or more species of polyatomic monovalent cation and one or more divalent cations, i.e. one or more species of divalent cation. It will be appreciated that these may dissociate in solution, and because of this, the number of cations associated with each nucleotide complex does not need to be a whole number, since in solution the ions may dissociate. The starting nucleotide complex may be considered to be a salt if the charges are entirely balanced.

This starting nucleotide complex may be as described in WO2021/161051 (PCT/GB2021/050366).

The starting nucleotide complex in solution is used to prepare a nucleotide complex with a reduced provision of monovalent cation. This is achieved by applying heat and/or a vacuum, such that the polyatomic moiety from the monovalent cations are rendered volatile and are removed from the solution. They are effectively evaporated. Also removed from the solution may be the solvent, for example water. This further reduced provision of monovalent cations provides a novel nucleotide complex, which does not require a buffering agent to re-solubilise.

In general, the inventors have mixed two different nucleotide complexes (one with solely divalent cation(s) and the other with solely monovalent cations) together in order to prepare the starting nucleotide complexes used in the method of the invention. Alternatively, these two nucleotide complexes can be mixed during or after the process to remove the monovalent entity. The nucleotide complex may each independently comprise a complex where not all of the negative charges are balanced. This has several advantages. Nucleotides complexed with divalent cations have a low solubility, and therefore are not in routine use for any application. Particular issues are seen with nucleotides complexed with magnesium ions; they are not in solution and are unusable in their current form. However, when mixed with nucleotide complexes associated with a monovalent cation or cations, the mixture is soluble and forms a solution. Therefore, this provides an elegant way of utilising a previously desired but unworkable nucleotide complex.

The polyatomic monovalent ion may be a sole species of a polyatomic ion, or a mixture of different species of polyatomic ion. The divalent ion may be a sole species of ion or a mixture of different species of ion. The divalent ion may be magnesium.

The polyatomic monovalent ion is rendered volatile under suitable conditions such as under heat and/or a vacuum, or with a change in pH. The polyatomic monovalent cation may therefore be a conjugate acid of a base, wherein the base is a volatile moiety. Thus, the deprotonated form of the polyatomic monovalent cation may be described as the volatile moiety. This may be referred to as the “polyatomic moiety”.

Volatility indicates the tendency of a moiety to evaporate under the given conditions, also described as how readily the moiety vaporises. This is indicated at a given temperature and pressure. Volatility is commonly described with reference to the boiling point of the moiety (for liquids in particular). Boiling point is the temperature at which the vapour pressure of a liquid is equal to the surrounding pressure, causing the liquid to rapidly evaporate, or boil. It is dependent on pressure. In general, the volatile moiety may have a boiling point of less than 100° C.

The drying process, removal or evaporation may be conducted under a vacuum. A vacuum may create a region with a gaseous pressure less than atmospheric pressure. Thus, the gaseous pressure may be any appropriate pressure ranging from 10⁵ Pascal (Pa) to 100 Pa. Thus, the pressure may be 10⁵ Pa, 10⁴ Pa, 10³ Pa, 10² Pa or 10¹ Pa or any pressure therebetween. A low vacuum may occur at 10⁵ to 3×10³ Pa, a medium vacuum at 3×10³ to 10⁻¹ Pa and a high vacuum at 10⁻¹ to 10⁻¹⁰ Pa. A low or medium vacuum is preferred. Standard atmospheric pressure is 101,325 Pa, and thus above the starting level for a low vacuum. A vacuum may be created using any appropriate technique, including the use of a vacuum pump or by any other method of reducing the pressure, such as the use of fast-flowing liquids. A vacuum may be created using any suitable laboratory equipment, aspirators, including traditional rotary vane pumps, combination pumps, piston pumps, diaphragm pumps and scroll pumps and oil-free, vacuum pumps. Vacuums are designed to reduce the pressure within a system thereby lowering the boiling point of a moiety. By reducing the boiling point, the moiety evaporates at a much lower temperature.

The drying process, removal or evaporation may be conducted at any appropriate temperature. The skilled person will be aware that the boiling point of the volatile moiety will determine a suitable set of conditions for drying, for example pressure and temperature. The process or method may be conducted at room temperature. Alternatively, heat may be applied to the starting nucleotide complex. The temperature may be raised to 25, 30, 35, 40, 45, 50, 55, 60, 65, 70, 75, 80, 85 or 90° C., or any temperature range therebetween. The temperature required to render the moiety volatile will depend upon the pressure under which the drying is performed.

It may be preferable that the suitable conditions are the application of heat and a vacuum. Apparatus is available for such combined treatments, as exemplified in the Examples.

Alternatively, or additionally, the moiety can be rendered volatile by altering the pH, such as increasing the pH by adding a basic component, for example.

The drying or treatment of the starting nucleotide complex under a vacuum may drive the equilibrium between the conjugate acid and volatile base to completion, such that substantially all of the volatile moiety is evaporated.

The drying or treatment of the starting nucleotide complex may result in substantially all of the polyatomic monovalent moiety being removed from the complex, such that less than 0.2 polyatomic moieties per nucleotide remain. Ideally, substantially all of the polyatomic moieties are removed. Without being bound by theory, the inventors consider that the nucleotide entity becomes protonated, leaving the volatile base free to evaporate. The nucleotide may be protonated on a nitrogen or phosphate group. Alternatively, a molecule of water is protonated, creating a hydronium ion.

The drying, removal or evaporation may result in the formation of a dried nucleotide complex, also described as a powder. As used herein, powder is a dry solid composed of many very fine particles that may flow freely when shaken or moved. The powder may be crystalline. The drying may result in the removal of substantially all of the solvent present in the starting nucleotide complex solution. In general, the solvent may be water. The water or other solvent may be evaporated during the drying process. Ideally, all of the solvent may be removed, such that the nucleotide complex produced is dry (0% starting volume). Alternatively, a residual amount of solvent may be left, such that the level of solvent in the product is approximately less than 10%, less than 9%, less than 8%, less than 7%, less than 6%, less than 5%, less than 4%, less than 3%, less than 2%, less than 1% starting volume. Such may be visually observed as a “sticky mass”. In the Examples, the nucleotide complexes were dried under a vacuum from a starting volume of greater than 1 ml to a final volume of around 20 μl.

Generally, a polyatomic monovalent cation is present at a ratio 0.2 to 4, or 0.2 to 2.5 polyatomic monovalent cations to the starting nucleotide complex according to the present invention. This range includes 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 and 2.5 or 2.6, 2.7, 2.8, 2.9, 3.0. 3.1, 3.2, 3.3, 3.4, 3.5, 3.6, 3.7, 3.8, 3.9 or 4 polyatomic monovalent ions per nucleotide complex. It is possible that the ions are shared between the nucleotide ions. These polyatomic moieties are effectively or substantially removed or evaporated using the drying process described herein. During the process of the invention, a proportion of the polyatomic moiety is removed. This proportion may be 60%, 65%, 70%, 80%, 85%, 90%, 95%, 96%, 97%, 98% or 99%. Preferably 100% Is removed. After the drying process, the level of polyatomic moiety (the volatile base of the conjugate acid) is greatly reduced, preferably to levels of less than 0.2 polyatomic moieties per nucleotide, less than 0.1 or even zero.

The process, termed a drying process described herein results in a nucleotide complex, then when re-suspended in water, has decreased in pH. This decrease in pH is thought to be due to the protonation of the nucleotide in the complex during the drying process. In the Examples, the inventors found that the resuspended nucleotide complexes were not as acidic as expected, and may be due to the ability of the nucleotide complex to act as a buffer. The inventors have not required the use of a buffer in order to re-solubilise or dissolve the nucleotide complex prior to use.

The polyatomic monovalent cations preferably include a protonated nitrogen atom. Suitable polyatomic monovalent cations include ammonium and derivatives thereof. It is this proton that is transferred to the nucleotide entity during vaporisation of the polyatomic moiety.

Once substantially or completely dried, the nucleotide complex may be stored in appropriate conditions, such as in a freezer, until required for use. In use, the prepared nucleotide complexes will require re-solubilisation, such as in water or another solvent. Such may be provided alone or in combination with a nucleotidyltransferase, to form a reaction mixture. Other components may be added to the reaction mixture.

The solvent for re-solubilisation may be water. The solvent may be any appropriate solvent. The inventors have surprisingly found that the re-solubilisation does not require a buffer, despite the loss of the volatile moiety. Thus, it is hypothesized that the nucleotide complex is capable of buffering the pH independently, potentially through the presence of one or more phosphate groups.

The re-solubilisation may permit the addition of other entities, such as the zwitterionic molecules hereinbefore described. These may be present in the solvent or added separately.

In the drying process or evaporation, the starting nucleotide complex may be at any appropriate concentration.

The advantage of using the drying process or evaporation is that it results in a novel nucleotide complex which is advantageous for use in the enzymatic synthesis of DNA in particular.

It is preferred that the concentration of nucleotides or complexes thereof in the enzymatic synthesis of DNA, i.e. in the reaction mixture, may be more than 30 mM and up to at least 160 mM. Such concentrations are important in the production of higher yields of DNA, which in the case of the two concentrations given can be as high as 9.75 g/l to 52 g/l. It is preferred that the concentration of nucleotide or complexes thereof stated is at the start of the synthesis reaction, i.e. is the starting or initial concentration of nucleotides or complexes thereof in the reaction mixture, which also includes the enzyme necessary for DNA synthesis. Subsequent addition of further components may reduce this concentration, and their use by the DNA synthesis enzyme will also reduce the concentration from the starting concentration. Those skilled in the art will be aware of how to calculate the concentration of nucleotides/nucleotide complexes as the process is prepared, based upon the volume of the other components and the stock nucleotide complex solution/powder used. As described above, it may be beneficial to keep the concentration of the nucleotide complexes in the DNA synthesis reaction constant, for example at 100 mM, 110 mM, 120 mM, 130 mM or 140 mM for the whole of the reaction, by progressively monitoring and adding further nucleotides when the concentration decreases, or by constant addition of nucleotide complex.

It should be noted that in the art of DNA synthesis or amplification, the term “nucleotide” is used when the author means “nucleotide salt”, since the provision and utilisation in DNA synthesis of nucleotides without any form of counter-ion is not currently possible.

The process may be a batch process or a continuous flow process. The batch may be a closed batch (i.e. all of the reaction components are provided at the start of the DNA synthesis) or further components can be supplied to the reaction as required during the process, such as described in WO2016/034849, incorporated herein by reference. Should further additions be required, this will dilute the concentration of the nucleotide or nucleotide complexes, unless further nucleotide complexes are added to replenish or increase the concentration.

The enzymatic cell-free synthesis of DNA with such ions can be carried out in minimal buffering agents, in which no additional salts which have been shown to enhance DNA synthesis or assist in primer binding, or detergents, are added. This minimal buffer may comprise an agent (a buffering agent) to stabilise the pH. The minimal buffering agent may contain a small amount of cations provided by the presence of a chemical used to denature the template, such as sodium, potassium or ammonium hydroxide. In the Examples, 5 mM sodium or potassium hydroxide was used as a denaturant, but this concentration may be altered to suit the conditions of the reaction, within the skill of those used to performing DNA denaturation, and amounts of sodium, potassium or ammonium hydroxide could be provided from 2.5 mM, up to 5 mM, up to 10 mM, 15 mM, 20 mM or 25 mM or more may be employed dependent upon the nature of the template. Therefore, the reaction mixture may contain a small or minimal amount of cations and anions which were not originally associated with the nucleotide complex.

Further advantages are described below.

### BRIEF DESCRIPTION OF THE DRAWINGS

The present invention will be described further below with reference to exemplary embodiments and the accompanying drawings, in which:

FIG. 1 is a plasmid map of the DNA template used in the Examples 1-3. Indicated are the various sequence components of the DNA template;

FIG. 2 is a plasmid map of the DNA template used in the Examples 4-6. Indicated are the various sequence components of the DNA template;

FIG. 3 is a graph depicting the yield from the DNA synthesis experiments conducted using the DNA template in Examples 2 and 3. This is a plot of nucleotide complex identity and concentration (mM) versus yield (g/L). Compared are the results for the reaction in water or with added buffering agents;

FIG. 4 is a graph depicting the peak yields obtained from the DNA synthesis experiments conducted using the DNA template in Example 4. This is a plot of nucleotide complex identity and concentration (mM) versus peak yield (g/L). These are the highest yields obtained over a range of incubation times;

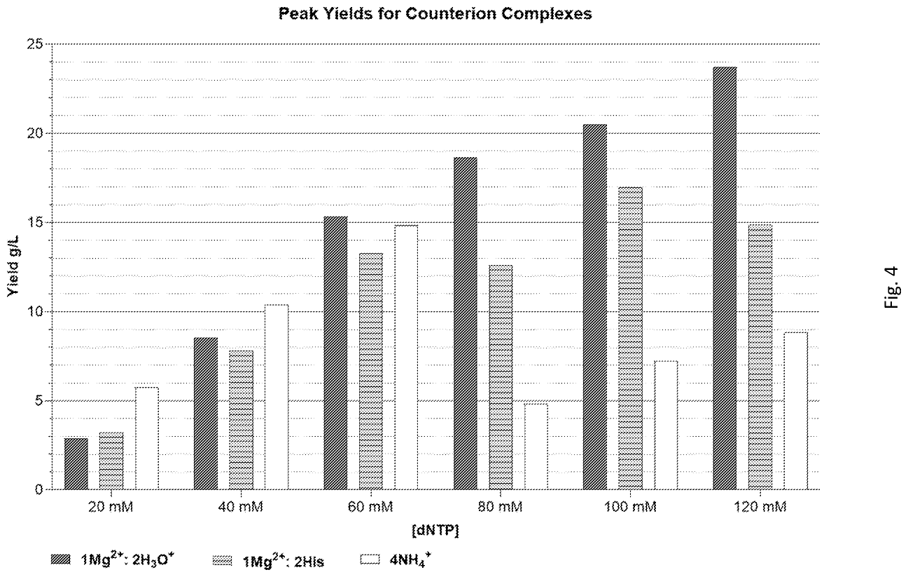

FIG. 5 is a graph depicting the yield obtained from the DNA synthesis experiments conducted using the DNA template in Example 5. This is a plot of nucleotide complex identity and incubation length (Day) versus yield (g/L). These are the yields obtained at 100 mM dNTP concentration over a range of incubation periods;

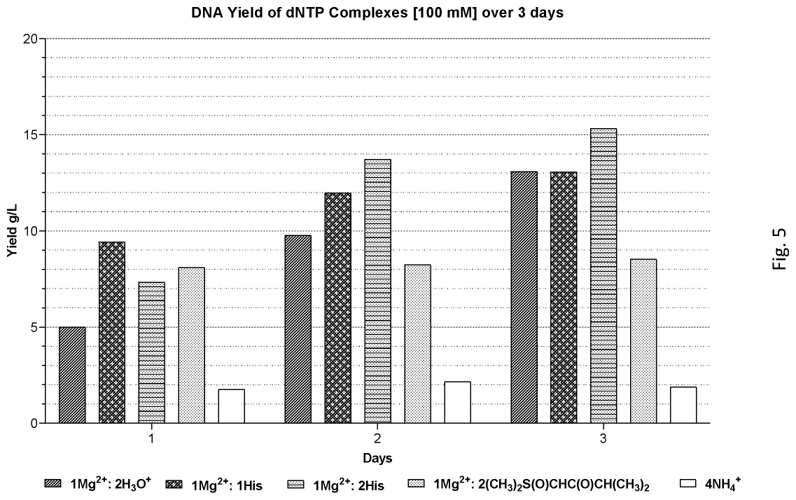

FIG. 6 is a graph depicting the yield obtained from the DNA synthesis experiments conducted using the DNA template in Example 5. This is a plot of nucleotide complex identity and incubation length (Day) versus yield (g/L). These are the yields obtained at 120 mM dNTP concentration over a range of incubation periods;

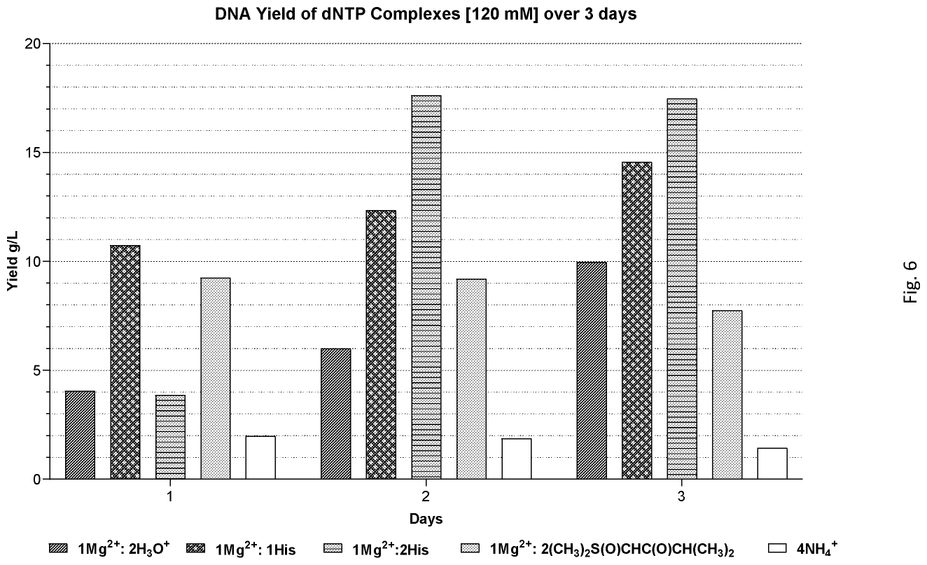

FIG. 7 is a graph depicting the yield obtained from the DNA synthesis experiments conducted using the DNA template in Example 6. This is a plot of nucleotide complex identity and concentration (mM) versus yield (g/L). These are the yields obtained after 5 days incubation with various nucleotide complexes; and

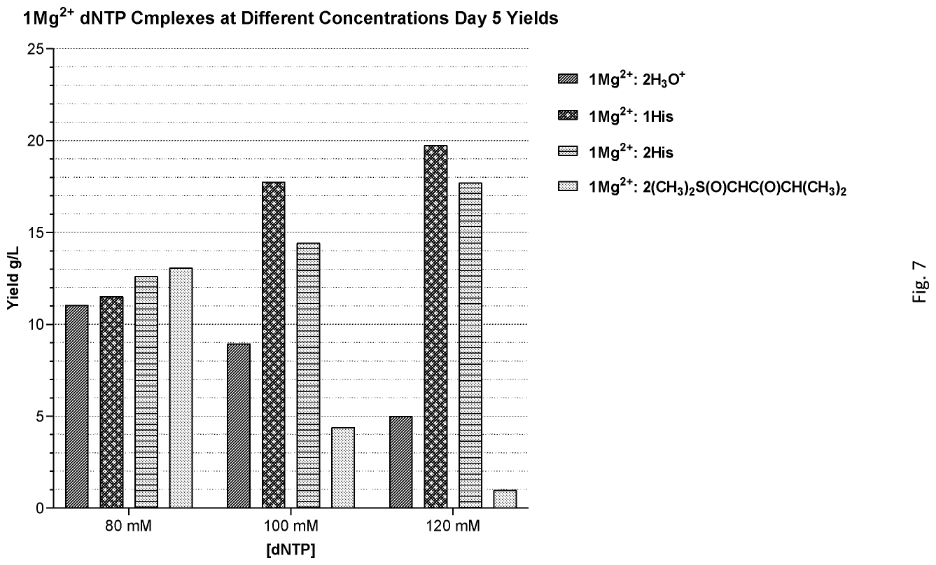

FIG. 8 is a graph depicting the yield obtained from the DNA synthesis experiments conducted using the DNA template in Example 6. This is a plot of nucleotide complex identity and concentration (mM) versus yield (g/L). These are the yields obtained after 10 days incubation with various nucleotide complexes.

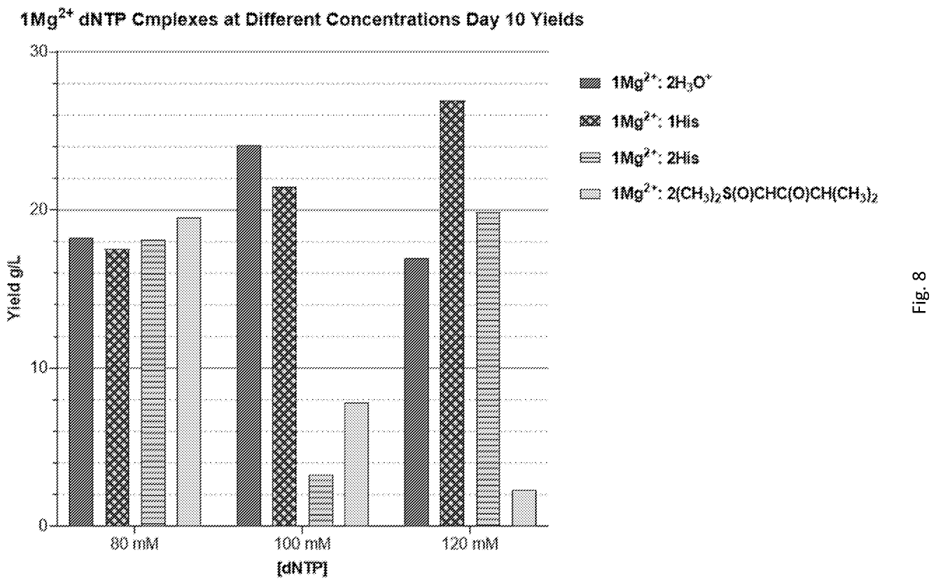

### DETAILED DESCRIPTION

The present invention relates to cell-free processes for large scale synthesis of DNA. The processes of the invention may allow for a high throughput synthesis of DNA.

The deoxyribonucleic acid (DNA) synthesised according to the present invention can be any DNA molecule. The DNA may be single stranded or double stranded. The DNA may be linear. The DNA may be processed to form circles, particularly minicircles, single stranded closed circles, double stranded closed circles, double stranded open circles, or closed linear double stranded DNA. The DNA may be allowed to form, or processed to form a particular secondary structure, such as, but not limited to hairpin loops (stem loops), imperfect hairpin loops, pseudoknots, or any one of the various types of double helix (A-DNA, B-DNA, or Z-DNA). The DNA may also form hairpins and aptamer structures.

The DNA synthesised may be of any suitable length. Lengths of up to or exceeding 77 kilobases may be possible using the processes of the invention. More particularly, the length of DNA which may be synthesised according to the processes of the invention may be in the order of up to 60 kilobases, or up to 50 kilobases, or up to 40 kilobases, or up to 30 kilobases. Preferably, the DNA synthesised may be 100 bases to over 77 kilobases, 500 bases to 60 kilobases, 200 bases to 20 kilobases, more preferably 200 bases to 15 kilobases, most preferably 2 kilobases to 15 kilobases.

The amount of DNA synthesised according to the processes of the present invention may exceed 9.75 g/l. It is preferred that the amount of DNA synthesised is greater than 3, 4, 5, 6, 7, 8, 9, 10, 11, 12, 13, 14, 15, 16, 17, 18, 19, 20, 21, 22, 23, 24, 25, 26, 27, 28, 29 or 30 g/l or more. A preferred amount of DNA synthesised is 5 g/l. The amount of DNA produced may be described as industrial or commercial quantities, on a large-scale or mass production. The DNA produced by the processes of the invention may be uniform in quality, namely in DNA length and sequence. The processes may thus be suitable for large-scale synthesis of DNA. The process may be uniform in terms of fidelity of synthesis.

Alternatively, the amount of DNA produced in the synthesis reaction may be compared to the theoretical maximum yield which would be achieved if 100% nucleotides were incorporated into the synthesised DNA. The methods of the invention not only improve the total yield obtained, but also the efficiency of the process, meaning that more of the supplied nucleotides are incorporated into the synthesised DNA product than in previous methods. Yields obtainable by the methods of the invention exceed 50% of the theoretical maximum, up to and exceeding 90% of the theoretical maximum. Therefore, the proportion of the theoretical maximum yield achieved by methods of the invention include 50%, 55%, 60%, 65%, 70%, 75%, 80%, 85%, 90% and 95% or greater. Conventionally, using commercially available nucleotide salts, yields achieved could be disappointing, due to effects of the ions that may be inhibitory to the process.

The DNA is synthesised in an enzymatic reaction. This enzymatic synthesis may involve the use of any DNA synthesising enzyme, a nucleotidyltransferase, capable of adding a nucleotide to a nascent polynucleotide chain, most notably a polymerase enzyme or a modified polymerase enzyme. These are discussed further below. The DNA synthesis may be de novo and not require a template. The enzymatic synthesis may also require the use of a template for the DNA synthesis. This template can be any suitable nucleic acid depending on the polymerase, but is preferably a DNA template.

The template may be any suitable template, merely providing the instructions for the synthesis of the DNA by including a particular sequence. The template may be single stranded (ss) or double stranded (ds). The template may be linear or circular. The template may include natural, artificial or modified bases or a mixture thereof.

The template may comprise any sequence, either naturally derived or artificial.

The template may be of any suitable length. Particularly, the template may be up to 60 kilobases, or up to 50 kilobases, or up to 40 kilobases, or up to 30 kilobases. Preferably, the DNA template may be 10 bases to 100 bases, 100 bases to 60 kilobases, 200 bases to 20 kilobases, more preferably 200 bases to 15 kilobases, most preferably 2 kilobases to 15 kilobases.

The template may be provided in an amount sufficient for use in the process by any method known in the art. For example, the template may be produced by PCR.

The whole or a selected portion of the template may be amplified in the process.

The template may comprise a sequence for expression. The DNA may be for expression in a cell (i.e. a transfected cell in vitro or in vivo), or may be for expression in a cell free system (i.e. protein synthesis). The sequence for expression may be for therapeutic purposes, i.e. gene therapy or a DNA vaccine. The sequence for expression may be a gene, and said gene may encode a DNA vaccine, a therapeutic protein and the like. The sequence may comprise a sequence which is transcribed into an active RNA form, i.e. a small interfering RNA molecule (siRNA). The sequence may comprise a sequence which is transcribed into mRNA, most particularly an mRNA for producing a vaccine. The sequence may comprise a sequence for the preparation of viral particles, such as lentivirus or adeno-associated virus.

If required, the template may be contacted with at least one polymerase, as described below.

The enzymatic DNA synthesis reaction may require at least one DNA synthesis enzyme (a nucleotidyltransferase). Preferably, the DNA synthesis enzyme is a polymerase. Polymerases link together nucleotides to form a DNA polymer. One, two, three, four or five different enzymes and/or polymerases may be used. The polymerase may be any suitable polymerase from any family of polymerases, such that it synthesises polymers of DNA. The polymerase may be a DNA polymerase. Any DNA polymerase may be used, including any commercially available DNA polymerase. Two, three, four, five or more different DNA polymerases may be used, for example one which provides a proofreading function and one or more others which do not. DNA polymerases having different mechanisms may be used e.g. strand displacement type polymerases and DNA polymerases replicating DNA by other methods. A suitable example of a DNA polymerase that does not have strand displacement activity is T4 DNA polymerase. Template-independent polymerases may be used, such as terminal transferases.

Modified polymerases may also be used. These may have been engineered to modify their characteristics, such as to remove their dependency upon a template, to change their temperature dependency or to stabilise the enzyme for use in vitro.

A polymerase may be highly stable, such that its activity is not substantially reduced by prolonged incubation under process conditions. Therefore, the enzyme preferably has a long half-life under a range of process conditions including but not limited to temperature and pH. It is also preferred that a polymerase has one or more characteristics suitable for a manufacturing process. The polymerase preferably has high fidelity, for example through having proofreading activity. Furthermore, it is preferred that a polymerase displays one or more of: high processivity, high strand-displacement activity and a low Kₘ for dNTPs and DNA. A polymerase may be capable of using circular and/or linear DNA as template. The polymerase may be capable of using dsDNA or ssDNA as a template. It is preferred that a polymerase does not display DNA exonuclease activity that is not related to its proofreading activity. Further, the polymerase may be capable of using an alternative nucleic acid as a template.

The skilled person can determine whether or not a given polymerase displays characteristics as defined above by comparison with the properties displayed by commercially available polymerases, e.g. Phi29 (New England Biolabs, Inc., Ipswich, MA, US), Deep Vent® (New England Biolabs, Inc.), Bacillus stearothermophilus (Bst) DNA polymerase I (New England Biolabs, Inc.), Klenow fragment of DNA polymerase I (New England Biolabs, Inc.), M-MuLV reverse transcriptase (New England Biolabs, Inc.), VentR® (exo-minus) DNA polymerase (New England Biolabs, Inc.), VentR® DNA polymerase (New England Biolabs, Inc.), Deep Vent® (exo-) DNA polymerase (New England Biolabs, Inc.) and Bst DNA polymerase large fragment (New England Biolabs, Inc.). Where a high processivity is referred to, this typically denotes the average number of nucleotides added by a polymerase enzyme per association/dissociation with the template, i.e. the length of nascent extension obtained from a single association event.

Strand displacement-type polymerases are preferred. Preferred strand displacement-type polymerases are Phi29, Deep Vent and Bst DNA polymerase I or variants of any thereof. “Strand displacement” describes the ability of a polymerase to displace complementary strands on encountering a region of double stranded DNA during synthesis. The template is thus amplified by displacing complementary strands and synthesising a new complementary strand. Thus, during strand displacement replication, a newly replicated strand will be displaced to make way for the polymerase to replicate a further complementary strand. The amplification reaction initiates when a primer or the 3′ free end of a single stranded template anneals to a complementary sequence on a template (both are priming events). When DNA synthesis proceeds and if it encounters a further primer or other strand annealed to the template, the polymerase displaces this and continues its strand elongation. The strand displacement may release single stranded DNA which can act as a template for more priming events. The priming of the newly released DNA may lead to hyper-branching, and a high yield of products. It should be understood that strand displacement amplification methods differ from PCR-based methods in that cycles of denaturation are not essential for efficient DNA amplification, as double-stranded DNA is not an obstacle to continued synthesis of new DNA strands. Strand displacement amplification may only require one initial round of heating, to denature the initial template if it is double stranded, to allow the primer to anneal to the primer binding site, if a primer is used. Following this, the amplification may be described as isothermal, since no further heating or cooling is required. In contrast, PCR methods require cycles of denaturation (i.e. elevating temperature to 94 degrees centigrade or above) during the amplification process to melt double-stranded DNA and provide new single-stranded templates. During strand displacement, the polymerase will displace strands of already synthesised DNA. Further, it will use newly synthesised DNA as a template, ensuring rapid amplification of DNA.

A strand displacement polymerase used in a process of the invention preferably has a processivity of at least 20 kb, more preferably, at least 30 kb, at least 50 kb, or at least 70 kb or greater. In one embodiment, the strand displacement DNA polymerase has a processivity that is comparable to, or greater than phi29 DNA polymerase.

Strand displacement replication is, therefore, preferred. During strand displacement replication, the template is amplified by displacing already replicated strands, which have been synthesised by the action of the polymerase, in turn displacing another strand, which can be the original complementary strand of a double stranded template, or a newly synthesised complementary strand, the latter synthesised by the action of a polymerase on an earlier primer annealed to the template. Thus, the amplification of the template may occur by displacement of replicated strands through strand displacement replication of another strand. This process may be described as strand displacement amplification or strand displacement replication.

A preferred strand displacement replication process is Loop-mediated isothermal amplification, or LAMP. LAMP generally uses 4-6 primers recognizing 6-8 distinct regions of the template DNA. In brief, a strand-displacing DNA polymerase initiates synthesis and 2 of the primers form loop structures to facilitate subsequent rounds of amplification. An inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. Modified LAMP procedures can also be adopted, where fewer internal primers are required.

A preferred strand displacement replication process is rolling circle amplification (RCA). The term RCA describes the ability of RCA-type polymerases to continuously progress around a circular DNA template strand whilst extending a hybridised primer. The “primer” may be added, created by a primase or generated by nicking one strand of a double stranded template. This amplification leads to formation of linear single stranded products with multiple repeats of amplified DNA. The sequence of the circular template (a single unit) is multiply repeated within a linear product. For a circular template, the initial product of strand displacement amplification is a single stranded concatamer, which is either sense or antisense, depending on the polarity of the template. These linear single stranded products serve as the basis for multiple hybridisation, primer extension and strand displacement events, resulting in formation of concatameric double stranded DNA products, again comprising multiple repeats of amplified DNA. There are thus multiple copies of each amplified “single unit” DNA in the concatameric double stranded DNA products. RCA polymerases are particularly preferred for use in the processes of the present invention. The products of RCA-type strand displacement replication processes may require processing to release single unit DNAs. This is desirable if single units of DNA are required. Typical strand displacement conditions using Phi29 DNA polymerase include high levels of magnesium ions, for example 10 mM magnesium (normally as a chloride salt) in combination with 0.2 to 4 mM nucleotides (when presented as typical lithium or sodium salts).

In order to allow for amplification, according to some aspects one or more primers may also be required by the enzymatic DNA synthesis. If no template is used, the primers allow for a starting point for DNA synthesis and are designed to begin the synthesis reaction. If a template is used, the primers may be non-specific (i.e. random in sequence) or may be specific for one or more sequences comprised within the template. Alternatively, a primase or modified polymerase enzyme may be supplied to generate the primer de novo. If the primers are of random sequence they allow for non-specific initiation at any site on the template. This allows for high efficiency of amplification through multiple initiation reactions from each template strand. Examples of random primers are hexamers, heptamers, octamers, nonamers, decamers or sequences greater in length, for example of 12, 15, 18, 20 or 30 nucleotides in length. A random primer may be of 6 to 30, 8 to 30 or 12 to 30 nucleotides in length. Random primers are typically provided as a mix of oligonucleotides which are representative of all potential combinations of e.g. hexamers, heptamers, octamers or nonamers in the template.

In one embodiment, the primers or one or more of the primers are specific. This means they have a sequence which is complementary to a sequence in the template from which initiation of amplification is desired. In this embodiment, a pair of primers may be used to specifically amplify a portion of the DNA template which is internal to the two primer binding sites. Alternatively, a single specific primer may be used. A set of primers may be employed.

Primers may be any nucleic acid composition. Primers may be unlabelled, or may comprise one or more labels, for example radionuclides or fluorescent dyes. Primers may also comprise chemically modified nucleotides. For example, the primer may be capped in order to prevent initiation of DNA synthesis until the cap is removed, i.e., by chemical or physical means. Primer lengths/sequences may typically be selected based on temperature considerations i.e. as being able to bind to the template at the temperature used in the amplification step. Primers may be RNA primers, such as those synthesized by a primase.

In certain aspects, the contacting of the template with the synthesis enzyme and one or more primers may take place under conditions promoting annealing of primers to the template. The conditions include the presence of single-stranded nucleic acid allowing for hybridisation of the primers. The conditions conventionally also include a temperature and buffer allowing for annealing of the primer to the template. Appropriate annealing/hybridisation conditions may be selected depending on the nature of the primer. An example of conventional annealing conditions, which may be used in the present invention include a buffer comprising 30 mM Tris-HCl pH 7.5, 20 mM KCl, 8 mM MgCl₂. In the Examples, the reactions with the nucleotide complexes of the invention are performed in 30 mM Tris pH 8.0 as a buffering agent alone. However, the present inventors have described conditions herein with reduced buffer and divalent metal ion components that still allow for primer binding and these are discussed further below. The annealing may be carried out following denaturation using heat followed by gradual cooling to the desired reaction temperature.

However, amplification using strand displacement replication can also take place without a primer, and thus requires no hybridisation and primer extension to occur. Instead, the single stranded template self-primes by forming hairpins, which have a free 3′ end available for extension. The remaining steps of the amplification remain the same. Alternatively, a double stranded template can be nicked to allow for strand displacement replication to use one strand of the template itself as a primer. Those skilled in the art are aware of all methods for providing initiation of amplification from a template.

The template and/or polymerase are also contacted with nucleotides, as nucleotide complexes as defined herein. The combination of template, nucleotidyltransferase and nucleotide complexes may be described as forming a reaction mixture. The reaction mixture may also comprise one or more primers or a primase. The reaction mixture may independently also include one or more divalent metal cations, should sufficient not be supplied with the nucleotide complexes. The reaction mixture may further comprise a chemical denaturant. Such denaturants can be potassium, ammonium or sodium hydroxide. The reaction mixture may further comprise additional enzymes, such as a helicase or a pyrophosphatase. The reaction mixture may contain pH buffering agents, and in some aspects, it contains no additionally added pH buffering agents.

A nucleotide is a monomer, or single unit, of nucleic acids, and nucleotides are composed of a nitrogenous base, a five-carbon sugar (ribose or deoxyribose), and at least one phosphate group. Any suitable nucleotide may be used.

The nucleotides are present as complexes, and are thus associated with divalent cations and a minimal amount, or lack of monovalent cations. Divalent cations are ionic species with a double positive charge, and may be a metal ion or a polyatomic ion.

A counter-ion is the ion that accompanies or is associated with an ionic species (the nucleotide in the present invention) in order to partially or completely balance the charge on the ionic species.

A complex is generally understood to be a molecular entity formed by loose association involving two or more component molecular entities (ionic or uncharged), or the corresponding chemical species. A complex may be either an ion or an electrically neutral molecule, formed by the union of simpler substances (as compounds or ions) and held together by forces that are chemical (i.e., dependent on specific properties of particular atomic structures) rather than physical. The bonding between the components is normally weaker than in a covalent bond.

The nucleotide complexes as prepared as described herein may include divalent cations. The divalent cations associated with the nucleotide in the complex may comprise one or more metals selected from the list consisting of: Mg²⁺, Be²⁺, Ca²⁺, Sr²⁺, Mn²⁺ or Zn²⁺, preferably Mg²⁺ or Mn²⁺. The ratio between the divalent metal cations and the nucleotide (nucleotide ion or nucleotide ionic species) may be about 1:1 in solution, but is preferably between 0.2:1 and 2:1, optionally 0.5:1 to 1.5:1. Ratios lower than 1:1 are desirable and are preferable in DNA synthesis since ratios higher than 1:1 may lead to some infidelity in DNA synthesis. The provision of the divalent cations in relation to the nucleotide complex may therefore reduce or remove the need to add additional divalent cations to the reaction mixture. However, should further be required, these divalent cations may be provided to the enzymatic DNA synthesis in the form of any suitable salt.

A divalent cation may be present at a ratio between 0.2 and 2 divalent cations pre nucleotide in the nucleotide complex. This range includes 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 and 2 divalent cations per nucleotide complex. Those skilled in the art will appreciate the non-whole numbers represent a sharing of the divalent ion between nucleotide free acids.

Optionally the nucleotide complex may include a nucleotide associated with a zwitterion molecule present at a ratio of between 0 and 4 zwitterion molecules per nucleotide. This range includes 0. 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 zwitterion molecules per nucleotide. Those skilled in the art will appreciate the non-whole numbers represent a sharing of the molecules between nucleotide free acids.

The nitrogenous base may be adenine (A), guanine (G), thymine (T), cytosine (C), and uracil (U). The nitrogenous base may also be modified bases, such as 5-methylcytosine (m5C), pseudouridine (L4), dihydrouridine (D), inosine (1), and 7-methylguanosine (m7G). The nitrogenous base may further be an artificial base. The concentration of nucleotide complexes may include any combination of the various nitrogenous bases.

It is preferred that the five-carbon sugar is a deoxyribose, such that the nucleotide is a deoxynucleotide.

The nucleotides may be in the form of deoxynucleoside triphosphate, denoted dNTP. This is a preferred embodiment of the present invention. Suitable dNTPs may include dATP (deoxyadenosine triphosphate), dGTP (deoxyguanosine triphosphate), dTTP (deoxythymidine triphosphate), dUTP (deoxyuridine triphosphate), dCTP (deoxycytidine triphosphate), dITP (deoxyinosine triphosphate), dXTP (deoxyxanthosine triphosphate), and derivatives and modified versions thereof. It is preferred that the dNTPs comprise one or more of dATP, dGTP, dTTP or dCTP, or modified versions or derivatives thereof. It is preferred to use a mixture of dATP, dGTP, dTTP and dCTP or modified version thereof. Any suitable ratios of these dNTPs can be used, according to the needs of the reaction.

The nucleotide complexes, may already be in solution prior to mixing with the nucleotidyltransferase, or may need to be supplied as a solid for example as a powder, and dispersed in solution. The nucleotide complexes may comprise modified nucleotides. The nucleotide complexes may be provided in a mixture of one or more suitable bases, preferably, one or more of adenine (A), guanine (G), thymine (T), cytosine (C). Two, three or preferably all four nucleotides (A, G, T, and C) are used in the process to synthesise DNA. These nucleotide complexes may all be present in substantially equal amounts or more of one or two may be provided, depending on the nature of the DNA to be synthesised.

The nucleotides may all be natural nucleotides (i.e. unmodified), they may be modified nucleotides that act like natural nucleotides and are biologically active (i.e. LNA nucleotides—locked nucleic acid), they may be modified and biologically inactive or they may be a mixture of unmodified and modified nucleotides, and/or a mixture of biologically active and biologically inactive nucleotides. Each type (i.e. base) of nucleotide may be provided in one or more forms, i.e. unmodified and modified, or biologically active and biologically inactive. All of these nucleotides are capable of forming appropriate complexes.

In one aspect of the invention, the nucleotides or nucleotide complexes are present at a concentration of at least 30 mM. According to this aspect, the nucleotides or nucleotide complexes may be present in the reaction mixture at a concentration of more than more than 30 mM, more than 35 mM, more than 40 mM, more than 45 mM, more than 50 mM, more than 55 mM, more than 60 mM, more than 65 mM, more than 70 mM, more than 75 mM, more than 80 mM, more than 85 mM, more than 90 mM, more than 95 mM, more than 100 mM, more than 110 mM, and more than 120 mM. Such concentrations are given as the concentration of nucleotide complex at the initiation or start of the process. The concentration is given after the addition of the nucleotide/nucleotide complexes, wherein the addition may be to the reaction mixture. The nucleotide complex may be any appropriate mixture of nucleotide complexes, with varying nitrogenous bases. The concentration applies to the sum total of nucleotide complexes present in at the start of the process, whatever their composition. Thus, for example, a 30 mM concentration of nucleotide salts may be any mixture of dCTP, dATP, dGTP and dTTP counter-ioned with appropriate monovalent and divalent cations.

It will be understood that nucleotides supplied as complexes may dissociate in water and other solvents to form an anionic nucleotide entity (nucleotide ion, nucleotide ionic species) and any associated cations and optionally zwitterionic (zwitterion) molecules, if present. As the nucleotide complex may be protonated due to the drying process, this may remain, or form a hydronium ion with any water molecules. According to the definitions used in the present invention, neither the proton nor hydronium ion are considered to be polyatomic monovalent cations as described herein.

It is a preferred part of any aspect of the present invention that the nucleotide complex is formed by a mixture of counter-ions and zwitterions.

The enzymatic DNA synthesis may be maintained under conditions promoting synthesis of DNA, and this will depend upon the particular method selected.

Amplification of a template via strand displacement is preferred. Preferably, the conditions promote amplification of said template by displacement of replicated strands through strand displacement replication of another strand. The conditions comprise use of any temperature allowing for amplification of DNA, commonly in the range of 20 to 90 degrees centigrade. A preferred temperature range may be about 20 to about 40 or about 25 to about 35 degrees centigrade. A preferred temperature for LAMP amplification is about 50 to about 70 degrees centigrade.

Typically, an appropriate temperature for enzymatic DNA synthesis is selected based on the temperature at which a specific polymerase has optimal activity. This information is commonly available and forms part of the general knowledge of the skilled person. For example, where phi29 DNA polymerase is used, a suitable temperature range would be about 25 to about 35 degrees centigrade, preferably about 30 degrees centigrade. However, a thermostable phi29 may operate at a higher constant temperature. The skilled person would routinely be able to identify a suitable temperature for efficient amplification according to the processes of the invention. For example, a process could be carried out at a range of temperatures, and yields of amplified DNA could be monitored to identify an optimal temperature range for a given polymerase. The amplification may be carried out at a constant temperature, and it is preferred that the process is isothermal. Since strand displacement amplification is preferred there is no requirement to alter the temperature to separate DNA strands. Thus, the process may be an isothermal process.

Other conditions promoting DNA synthesis are conventionally thought to comprise the presence of suitable buffering agents/pH and other factors which are required for enzyme performance or stability. Suitable conventional conditions include any conditions used to provide for activity of polymerase enzymes known in the art.

For example, the pH of the reaction mixture may be within the range of 3 to 10, preferably 5 to 8 or about 7, such as about 7.5. Whilst not necessary for the use of the novel nucleotide complexes, pH may be maintained in this range by use of one or more buffering agents (also called pH buffering agents). The function of a buffering agent is to prevent a change in pH. Such buffers (buffering agents) include, but are not restricted to MES, Bis-Tris, ADA, ACES, PIPES, MOBS, MOPS, MOPSO, Bis-Tris Propane, BES, TES, HEPES, DIPSO, TAPSO, Trizma, HEPPSO, POPSO, TEA, EPPS, Tricine, Gly-Gly, Bicine, HEPBS, TAPS, AMPD, TABS, AMPSO, CHES, CAPSO, AMP, CAPS, CABS, phosphate, citric acid-sodium hydrogen phosphate, citric acid-sodium citrate, sodium acetate-acetic acid, imidazole and sodium carbonate-sodium bicarbonate. Preferred are buffering agents that do not provide further cations to the reaction mixture, nor complex with metal cations present in the reaction mixture as discussed previously.

A buffer is generally defined by a mixture of reaction components. Usually included is a buffering agent to maintain a stable pH; one or more additional salts composed of a cationic and anionic species i.e. sodium chloride, potassium chloride; and/or detergents such as Triton-X-100 to ensure optimal activity or stability of the enzymes. A minimal buffer is composed of only a buffering reagent with no additional salts or detergents provided, with the proviso that small amounts of cationic species may be present for DNA synthesis in which chemical denaturation is required. Surprisingly, using higher concentrations of nucleotide salts in the processes of the invention permits the use of these minimal buffers.

A “no buffer” system lacks a provided or defined pH buffering agent in the mixture of reaction components and lacks additional salts or detergents. This “no added buffering agent” system contains only the reaction components required for the DNA synthesis alone, and contains cationic species provided for chemical denaturation only (if required). Thus, in this system, there are no additional ions added beyond those that serve a specific purpose in the DNA synthesis reaction. The counter-ions provided with the nucleotides (as a complex) serve to stabilise the nucleotide prior to use in the process.

While the application of heat (exposure to 95° C. for several minutes) is used to denature double stranded DNA other approaches may be used which are more suitable for DNA synthesis. Double stranded DNA can be readily denatured by exposure to a high or low pH environment or where cations are absent or present in very low concentrations, such as in deionized water. The polymerase requires the binding of a short oligonucleotide primer sequence to a single stranded region of the DNA template to initiate its replication. The stability of this interaction and therefore the efficiency of DNA synthesis may particularly be influenced by the concentration of metal cations and particularly divalent cations such as magnesium (Mg²⁺) ions which may be seen as an integral part of the process.

The enzymatic DNA synthesis may also require the presence of additional divalent metal ions, namely divalent cations that are supplied externally to the nucleotide complex. The process may comprise the use of salts of divalent metal ions: magnesium (Mg²⁺), manganese (Mn²⁺), calcium (Ca²⁺), beryllium (Be²⁺), zinc (Zn²⁺) and strontium (Sr²⁺). The most often used divalent ions in DNA synthesis is magnesium or manganese, since these act as a cofactor in DNA synthesis. Any suitable anion may be utilised in such salts, whilst being mindful that the choice of anion can have an effect on the pH of the reaction mixture, and should be suitably accounted for.

Detergents may also be included in the reaction mixture in certain aspects. Examples of suitable detergents include Triton X-100™, Tween 20™ and derivatives of either thereof. Stabilising agents may also be included in the reaction mixture. Any suitable stabilising agent may be used, in particular, bovine serum albumin (BSA) and other stabilising proteins. Reaction conditions may also be improved by adding agents that relax DNA and make template denaturation easier. Such agents include, for example, dimethyl sulphoxide (DMSO), formamide, glycerol and betaine. DNA condensing agents may also be included in the reaction mixture. Such agents include, for example, polyethylene glycol or cationic lipid or cationic polymers.

However, in certain embodiments, these components may be reduced or removed from the reaction mixture, for example in the minimal or no added buffering agent systems.

It should be understood that the skilled person is able to modify and optimise synthesis conditions for the processes of the invention using these additional components and conditions on the basis of their general knowledge. Likewise the specific concentrations of particular agents may be selected on the basis of previous examples in the art and further optimised on the basis of general knowledge.

As an example, a suitable reaction buffer used in RCA-based methods in the art is 50 mM Tris HCl, pH 7.5, 10 mM MgCl₂, 20 mM (NH₄)₂SO₄, 5% glycerol, 0.2 mM BSA, 1 mM dNTPs. A preferred reaction buffer used in the RCA amplification is usually 30 mM Tris-HCl pH 7.9, 30 mM KCl, 7.5 mM MgCl₂, 10 mM (NH₄)₂SO₄, 4 mM DTT, 2 mM dNTPs. This buffer is particularly suitable for use with Phi29 DNA polymerase when conventional nucleotides are purchased.

A suitable reaction buffer for use with the nucleotide complexes of the invention is 60 mM Tris pH 8.0. A further suitable reaction buffer is 30 mM Tris pH 8.0. Alternative conditions include 30 mM Tris HCl, pH 7.9, 5 mM (NH₄)₂SO₄, and 30 mM KCl. Under certain circumstances, the enzymatic DNA synthesis may be conducted in water (“no added buffering agent”).

The enzymatic DNA synthesis may also comprise the use of one or more additional proteins. The template may be amplified in the presence of at least one pyrophosphatase, such as Yeast Inorganic pyrophosphatase. Two, three, four, five or more different pyrophosphatases may be used. These enzymes are able to degrade pyrophosphate generated by the polymerase from dNTPs during strand replication. Build-up of pyrophosphate in the reaction can cause inhibition of DNA polymerases and reduce speed and efficiency of DNA amplification. Pyrophosphatases can break down pyrophosphate into non-inhibitory phosphate. An example of a suitable pyrophosphatase for use in the processes of the present invention is Saccharomyces cerevisiae pyrophosphatase, available commercially from New England Biolabs, Inc.

Any single-stranded binding protein (SSBP) may be used in the processes of the invention, to stabilise single-stranded DNA. SSBPs are essential components of living cells and participate in all processes that involve ssDNA, such as DNA replication, repair and recombination. In these processes, SSBPs bind to transiently formed ssDNA and may help stabilise ssDNA structure. An example of a suitable SSBP for use in the processes of the present invention is T4 gene 32 protein, available commercially from New England Biolabs, Inc.

The yield of the reaction relates to the amount of DNA synthesised. The expected yield from a process according to the present invention may exceed 3 g/l. It is preferred that the amount of DNA synthesised is greater than 3, 4, 5, 6, 7, 8, 9, 10, 11, 12, 13, 14, 15, 16, 17, 18, 19, 20, 21, 22, 23, 24, 25, 26, 27, 28, 29 or 30 g/l or more. A preferred amount of DNA synthesised is 5 g/l. 30 mM nucleotide complex is capable of generating 9.74 g/l DNA. The present invention improves the yield possible from enzymatic synthesis of DNA. It is an object of the present invention to improve the yield of a cell-free enzymatic DNA synthesis process, such that DNA can be synthesised on a large scale in a cost-effective way. The present invention allows the manufacture/synthesis of DNA economically on an industrial scale using an enzymatic process catalysed by a DNA synthesis enzyme or polymerase. The present process allows the efficient incorporation of nucleotides into the DNA product. It is thought that the processes of the invention will allow reaction mixtures to be scaled up into several litres, including tens of litres. The improved yield, productivity or processivity may be compared to an identical reaction mixture where all of the nucleotides are supplied as conventional salts with monovalent cation counter-ions (generally lithium or sodium).

In one embodiment, the present invention relates to a process for enhancing the synthesis of DNA. This enhancement may be compared to an identical reaction mixture, with the exception that all of the nucleotides complexes used are exclusively monovalent cation counter-ions, or a mixture thereof. The process may involve the use of a novel nucleotide complex prepared as described herein.

In one aspect, the invention provides a cell-free process for synthesising DNA comprising contacting a DNA template with at least one nucleotidyltransferase in the presence of one or more nucleotide complexes, wherein each of said nucleotide complexes are associated with between 0.2 and 2 divalent cations and between 0 and 1 monovalent cations, preferably less than 0.2 monovalent cations. The monovalent cations are preferably polyatomic monovalent cations, optionally containing a Nitrogen atom. Optionally the nucleotide complex may also be associated with between 0 and 4 zwitterion molecules per nucleotide.

It is preferred that the concentration of nucleotides referred to herein for the synthesis of DNA is the starting concentration of nucleotides at the start of the process, the initial concentration when the reaction mixture is formed.

The invention may also relate to a cell-free process for synthesising DNA comprising contacting a DNA template with at least one nucleotidyltransferase in the presence of one or more nucleotide complexes in a concentration of over 30 mM. The invention provides a cell-free process for the enzymatic synthesis of DNA comprising the use of nucleotides supplied as complexes, wherein said each of said complexes are a nucleotide associated with between 0.2 and 1.5 divalent cations and between 0.2 and 1 polyatomic monovalent cations, optionally less than 0.2, preferably wherein the nucleotide complexes are obtained, supplied or are present in a concentration greater than 30 mM. Optionally the nucleotide complex may also be associated with between 0 and 4 zwitterion molecules per nucleotide.

The nucleotide complexes may be present at a concentration of at least 40 mM, 50 mM, 60 mM, 70 mM, 80 mM, 90 mM, 100 mM, 110 mM or 120 mM. Optionally the complexes are present at a concentration of 40 mM to 160 mM, 50 mM to 150 mM, 60 mM to 140 mM, 70 mM to 130 mM or 80 mM to 120 mM, or any range between these values.

The invention further provides an enzymatic DNA synthesis which is performed under conditions of reduced or even no further additionally supplied divalent cations, preferably magnesium, comprising the use of nucleotide complexes, wherein each of said complexes comprise a nucleotide associated with between 0.2 and 1.5 divalent cations and between 0 and 1, optionally less than 0.2 monovalent cations. The provision of the divalent cation in the nucleotide complex avoids the further use of divalent cation salts in the process. However, in certain circumstances, the amount of divalent cation salts such as magnesium, are reduced using the complexes of the invention.

A used herein, those skilled in the art will appreciate that the novel nucleotide complex comprise, in terms of ionic entities, substantially only those listed with the complex. Alternatively worded, the novel nucleotide complexes consist essentially of the listed entities.

### CLAUSES

A. A process for the preparation of a nucleotide complex comprising drying a nucleotide complex which comprises a nucleotide associated with between 0.2 and 1.5 divalent cations and between 0.2 and 2.5 polyatomic monovalent cations per nucleotide, wherein the polyatomic monovalent cations are rendered volatile under vacuum.
B. The process of clause A wherein the polyatomic monovalent cation is the conjugate acid of a base, wherein the base is a volatile moiety.
C. The process of clause A or clause B, wherein a deprotonated form of the polyatomic monovalent cation is a volatile moiety.
D. The process of any preceding clause wherein the drying is performed at room temperature, or with the application of heat.
E. The process of any preceding clause wherein the drying is performed under a vacuum.
F. The process of any preceding clause wherein the drying is performed under a pressure of 10⁵ Pa than to 10⁻¹⁰ Pa.
G. The process of any preceding clause, wherein substantially all of the polyatomic monovalent cations are rendered volatile and are removed in the drying process.
H. The process of any preceding clause wherein substantially all of the polyatomic monovalent cations form a volatile moiety and are removed in the drying process.
I. The process of any preceding clause, wherein there is a resultant decrease in pH once the nucleotide complex is resuspended in water.
J. The process according to any preceding clause wherein the divalent cation is selected from any one or more of: magnesium, manganese or calcium.
K. The process according to any one preceding clause wherein the divalent cation is present at a ratio of 1:1 to the nucleotide.
L. A process as claimed in any preceding clause wherein the monovalent cations are rendered volatile under vacuum, and resultant volatile moieties have a boiling point lower than 100° C. at standard atmospheric pressure.
M. A process as claimed in any preceding clause wherein said polyatomic monovalent cations comprise a nitrogen atom, optionally comprising a protonated nitrogen atom.
N. A process as claimed in any preceding clause wherein said polyatomic monovalent cation is an ammonium salt or an ionic derivative thereof.
O. The process according to any preceding clause wherein the dried nucleotide complex is re-suspended in a solvent.
P. The process according to claim G wherein the nucleotide complex can be re-suspended in the absence of a buffering agent.
Q. The process of any preceding clause wherein the nucleotide complex is dried to obtain a powder.
R. A nucleotide complex produced by the process of any preceding clause.
S. A cell-free process for the enzymatic synthesis of DNA in solution comprising obtaining a nucleotide complex as claimed in clause R and adding a nucleotidyltransferase.
T. A process, nucleotide complex or cell-free process to any preceding clause wherein the nucleotide complex is soluble.
U. A cell-free process according to clause S or T wherein said nucleotide complex is obtained at a concentration of at least 80 mM, optionally at least 100 mM.
V. A cell-free process according to any to any one of clauses S to U wherein said nucleotide complex and nucleotidyltransferase form a reaction mixture.
W. A cell-free process according to any one of clauses S to V wherein further components are added to the reaction mixture, including but not limited to any one or more of the following:
   a) template nucleic acid;
   b) primer;
   c) primase;
   d) denaturing agent, such as sodium or ammonium hydroxide;
   e) buffering agents; including buffering salts;
   f) pyrophosphatase; and/or
   g) magnesium or manganese salts
X. A cell-free process according to clause W wherein magnesium or manganese salts are added to the reaction mixture as a co-factor for the nucleotidyltransferase, such that the total ratio of the magnesium and/or manganese to nucleotide does not exceed 2:1.
Y. A cell-free process according to any one of clauses S to X wherein said nucleotidyltransferase is a DNA polymerase, preferably a strand-displacing DNA polymerase.
Z. A cell-free process according to clause Y wherein said nucleotidyltransferase is capable of isothermal DNA synthesis.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments, which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

The invention will now be described with reference to several non-limiting examples.

### EXAMPLES

### Materials and Methods

### Materials and Methods

### Reagents

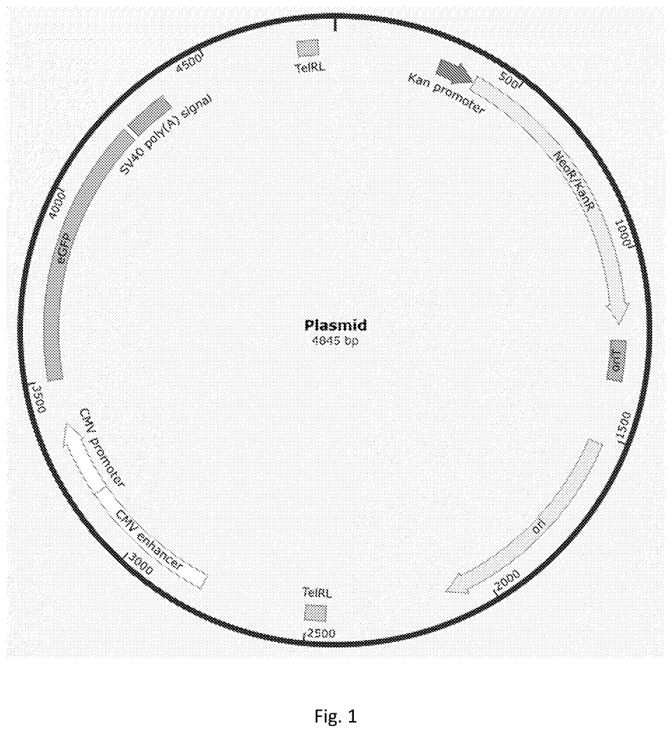

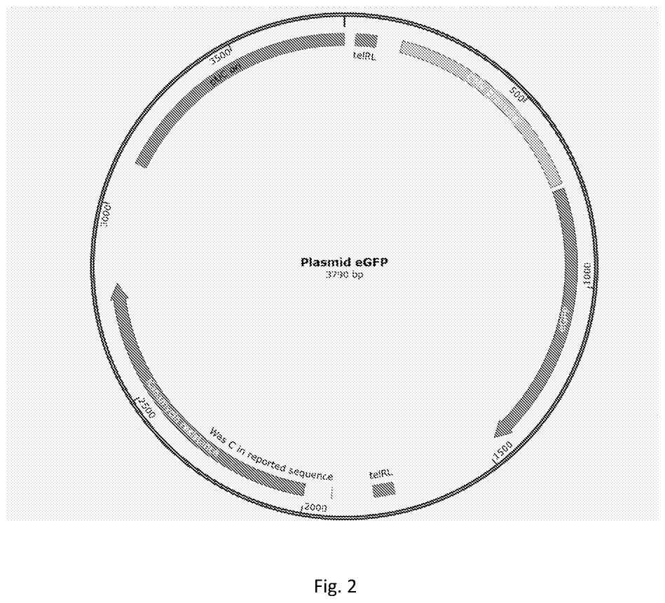

The following reagents supplied were used in the presented examples:
Solution 1-200 mM dATP: 4NH₄⁺
Solution 2-200 mM dCTP: 4NH₄⁺
Solution 3-200 mM dGTP: 4NH₄⁺
Solution 4-200 mM dTTP: 4NH₄⁺
Solution 5-66 mM dATP: 2Mg²⁺
Solution 6-59 mM dCTP: 2Mg²⁺
Solution 7-64 mM dGTP: 2Mg²⁺
Solution 8-74 mM dTTP: 2Mg²⁺
Phi29 DNA polymerase, stock concentration 1.6 g/L or 0.8 g/L (produced in-house)
Thermostable pyrophosphatase, stock concentration 2000 U/mL (NEB)
DNA primer, stock concentration 5 mM (Oligofactory)
Plasmid template: eGFP CMV, as shown in FIG. 1 stock concentration 1.393 g/L (produced in house)—Examples 2 and 3
ProTLx-K 15-10-15-0-15 eGFP B5x4 iAMB2016 DS3763 as shown in FIG. 2, stock concentration 595 ng/uL (produced in house)—Examples 4 to 6
Nuclease free water (Sigma Aldrich)
5M NaOH (Sigma Aldrich)
PEG 8000 (Applichem)
Tris-Base (Thermo Fisher Scientific)
Tris-HCl (Sigma Aldrich)
NaCl (Sigma Aldrich)
Dimethylsulfoxonium-(isobutanoyl)methylide—C₇H₁₄O₂S (later referred to as simply Ylide) (Merck Life Science)
L-Histidine (Thermo Fisher Scientific)

### Example 1

Preparation of dNTP Mixes According to the Present Invention:
For ammonium (dNTPs: 4 NH₄⁺) complexes used as a control, the individual dNTPs (sol 1 to 4) were mixed 1:1:1:1 to produce a stock concentration of 200 mM dNTP mixture: the Mixes were stored at −20° C.
For the magnesium mixed complexes (dNTPs: NH₄⁺/Mg²⁺), dNTPs (sol 1 to 4 and 5 to 8) were mixed in such a way to provide an equimolar amount of each specific nucleotide (i.e. dATP, dCTP, dGTP & dTTP): the Mixes were prepared and stored at −20° C.

TABLE 1
Ammonium dNTP Complex

| dNTP | [dXTP] mM | Volume (μL) |
|---|---|---|
| dATP (sol 1) | 200 | 1000 |
| dCTP (sol 2) | 200 | 1000 |
| dGTP (sol 3) | 200 | 1000 |
| dTTP (sol 4) | 200 | 1000 |

TABLE 2

| Magnesium dNTP Complex | | | Ammonium dNTP Complex | | |
|---|---|---|---|---|---|
| dNTP | [dXTP] mM | Volume (μL) | dNTP | [dXTP] mM | Volume (μL) |
| dATP (sol 5) | 66 | 1515 | dATP (sol 1) | 200 | 500 |
| dCTP (sol 6) | 59 | 1695 | dCTP (sol 2) | 200 | 500 |
| dGTP (sol 7) | 64 | 1563 | dGTP (sol 3) | 200 | 500 |
| dTTP (sol 8) | 74 | 1351 | dTTP (sol 4) | 200 | 500 |

For 200 mM 4NH₄⁺ dNTPs—Ammonium nucleotides were mixed in such a way to provide equimolar amounts of each nucleotide as detailed in Table 1. The final volume of 4000 μL mixed dNTPs resulted in 200 mM of 4NH₄⁺ dNTPs.
For 200 mM 1Mg²⁺:2NH₄⁺ dNTPS—Magnesium nucleotides were mixed in such a way to provide equimolar amounts of each nucleotide. The volumes correspond to 100 mM of each nucleotide such if the final volume was 4000 μL. To make Ammonium:Magnesium (1Mg²⁺:2NH₄⁺) dNTPs, the final volume of 6124 μL of Magnesium dNTPs was reduced to powder (i.e. 0 μL) by 60° C. speedvac. The powder was resuspended using the Ammonium pre-mixed nucleotides as detailed in Table 2 with an additional 2000 μL water to a final volume of 4000 μL resulting in 200 mM 1Mg²⁺:2NH₄⁺ dNTPs that were stored at −20° C. for later use. These steps were required because the 2Mg dNTPs are insoluble at higher concentrations (100 mM) but are more soluble at lower concentrations. Therefore, soluble nucleotide salts at a lower concentration were concentrated by drying prior to mixing with ammonium-ioned dNTPs to form the starting material for making the new nucleotide complexes, or used as a comparison.
For 200 mM 1Mg²⁺ dNTPs—previously prepared Ammonium:Magnesium (200 mM 1Mg²⁺:2NH₄⁺) dNTPs were split into four 1000 μL aliquots in 15 mL falcon tubes and either reduced to a sticky, solid-like pellet (about 20 μl) by 60° C. speedvac or dried to a powder (0 μl). Each pellet or powder was resuspended in 1000 μL of water, resulting in 200 mM 1Mg²⁺ dNTPs. Examples 2 and 3 were prepared using the nucleotide complexes prepared as far as a 20 μl pellet.

For 200 mM 1Mg²⁺ dNTPs—previously prepared Ammonium: Magnesium (200 mM 1Mg²⁺:2NH₄^{+) dNTPs were split into} 1000 μL aliquots in 2 mL Eppendorf tubes and either reduced to a sticky, solid-like pellet (about 20 μl) by 60° C. speedvac or dried to a powder (0 μl). Each pellet or powder was resuspended in 1000 μL of water, resulting in 200 mM 1Mg²⁺ dNTPs.

For 200 mM 1Mg²⁺ 1His, 200 mM 1Mg²⁺ 2His dNTPs and 200 mM 1Mg²⁺ 2Ylide dNTPs-previously prepared Ammonium:Magnesium (200 mM 1Mg²⁺:2NH₄⁺) dNTPs were split into 1000 μL aliquots in 2 mL Eppendorf tubes and reduced to a sticky, solid-like pellet (about 20 μl) by 60° C. speedvac. Each pellet was resuspended in 1000 μL of 200 mM L-Histidine (Fisher Scientific), 400 mM L-Histidine (Fisher Scientific), or 400 mM Ylide, resulting in 200 mM 1Mg²⁺ 1His, 200 mM 1Mg²⁺ 2His dNTPs and 200 mM 1Mg²⁺ 2Ylide dNTPs, respectively.

### Drying of the Nucleotide Complex to Remove Ammonia

The nucleotide complexes (1Mg:2NH4) prepared above were placed into a SpeedVac for drying under a vacuum, The Speedvac used was Eppendorf Concentrator Plus with a vacuum of 20 hPa (20 mbar), temperature of 60° C. and a fixed speed of 1400 rpm. All samples were dried over a space of 5-12 hours or 5-24 hours until the required level of solvent remained.
pH of dNTP Mixes
The pH of each dNTP mix was measured before and after the drying procedure to document the physical effect of evaporating the volatile entity during the drying process. The pH of a nucleotide salt with ammonium is given as a comparison:

TABLE 3

| dNTP complex | pH |
|---|---|
| 4NH₄ dNTPs | 7.564 |
| 1Mg:2NH₄ dNTP complexes | 6.531 |
| (starting material to make | |
| the nucleotides of the | |
| invention) | |
| 1Mg dNTP complexes | 5.952-6.023 |
| prepared according to the | |
| process of the invention | |
| 1Mg²⁺ 2His dNTP complexes | 7.490 |
| prepared according to the | |
| process of the invention | |

It can be seen that after drying, the pH of the nucleotide complex has decreased, indicating that that ammonia has been released from the nucleotide complex and evaporated.

### Example 2

### DNA Amplification Reaction Setup

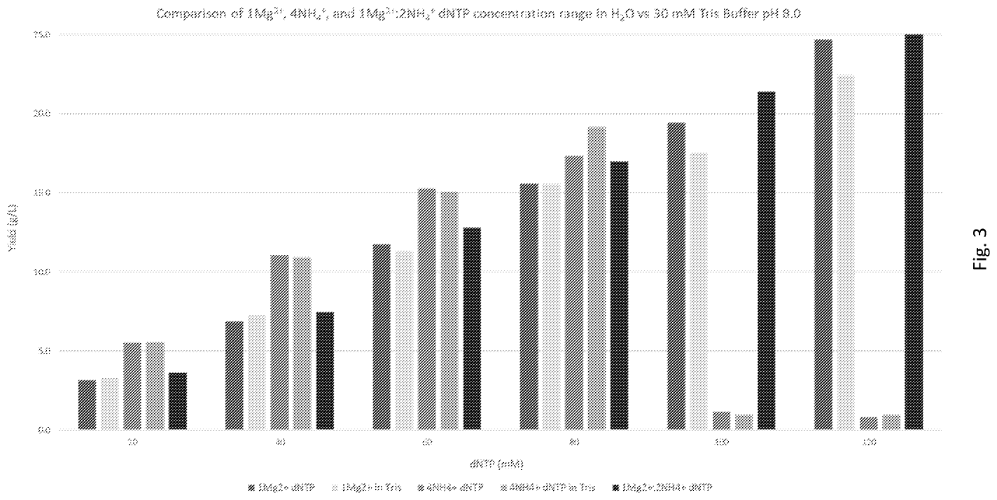

The various nucleotides prepared according to Example 1 were tested in a DNA amplification reaction to demonstrate whether such complexes could be used with a DNA polymerase and to determine the advantages of using such a complex in the preparation of DNA. As a comparison, two other nucleotide complexes were used. One comparator is a starting material from which the nucleotide complexes are prepared according to the present invention (1Mg: 2NH₄ dNTP). The other comparator is a nucleotide complex with 4 ammonium ions (4 NH₄). The 4 NH₄ comparator was used more extensively in the Examples presented herein.
Reactions were set up at a 100 μl or 200 μL scale as follows: A Denaturation Mix was prepared and left at room temperature for 15 minutes while the Reaction Mix was assembled. These were then mixed, and the DNA polymerase and pyrophosphatase added. DNA amplification experiments were performed using various dNTPs at a range of dNTP concentrations. Reactions were stopped after a suitable period of time, such as 168 hours, and samples were immediately processed as detailed below. Other period of times evaluated were 48 hours, 96 hours, 216 hours and 480 hours.
For the dNTP complexes lacking magnesium, an equimolar amount of magnesium salt, specified below, was added to the Reaction Mix. For the dNTPs complexes including magnesium a (i.e. 1Mg), as described above, no additional magnesium was supplied to the Reaction Mix. Table 4 shows the experimental protocol reaction setup for the dNTPs without magnesium (4NH₄*) while Table 5 shows the reaction setup for the magnesium complexed dNTPs (1Mg²⁺, 1Mg²⁺:2NH₄⁺ and 1Mg²⁺:2 His).
The experiments were carried out to determine if the ammonium could be made volatile and removed from the nucleotide complex, resulting in a nucleotide complex that was still functional in a DNA synthesis reaction, in particular a rolling circle amplification reaction. The materials and methods for RCA are described above. Reactions were allowed to proceed for the specified time period (168 hours) at a temperature of 30° C. followed by processing and quantification.
The Effects of Different Nucleotide Complexes and their Concentration on DNA Synthesis
This details the experimental set up for the data depicted in FIG. 3.

TABLE 4
DNA synthesis by Rolling Circle Amplification (RCA) Reaction components
for examining the effects of various nucleotide complexes: for these experiments 1Mg²⁺
dNTPs, 1Mg²⁺ 2Nh₄ dNTPS of 4NH₄ dNTPs

| | | Stock | | | | Final reaction | |
|---|---|---|---|---|---|---|---|
| | Reagent | concentration | | Volume | | concentration | |
| Denaturation | Plasmid | 1.393 | g/L | 0.29 | μL | 2 | ng/μL |
| Mix | Template | | | | | | |
| | NaOH | 1M | | 2.0 | μL | 10 | mM |
| | DNA primer | 5 | mM | 2.0 | μL | 50 | μM |
| | water | | | 7.71 | μL | to 12 μL | |
| Reaction Mix | Nucleotide | 200 | mM | Variable | | Variable- as shown | |
| | complex | | | | | | |
| | MgCl₂ | 2000 | mM | Variable | | Equimolar to dNTP | |
| | | | | | | molar conc. | |
| | water | | | 103.48 | μL | to 187.48 μL total | |
| Enzyme 1 | Pyrophosphatase | 2000 | U/mL | 0.2 | μL | 2 | U |
| Enzyme 2 | Phi29 | 1.6 | g/L | 0.32 | μL | 2.56 | mg/L |

DNA polymerase

TABLE 5
DNA synthesis by Rolling Circle Amplification (RCA) Reaction components
for examining the effects of the new nucleotide complexes. For these experiments 1Mg²⁺
dNTPs, 1Mg²⁺ 2Nh₄ dNTPS of 4NH₄ dNTPs

| | | Stock | | | | Final reaction | |
|---|---|---|---|---|---|---|---|
| | Reagent | concentration | | Volume | | concentration | |
| Denaturation | Plasmid | 1.393 | g/L | 0.29 | μL | 2 | ng/μL |
| Mix | Template | | | | | | |
| | NaOH | 1M | | 2.0 | μL | 10 | mM |
| | DNA primer | 5 | mM | 2.0 | μL | 50 | μM |
| | water | | | 7.71 | μL | to 12 μL | |
| Reaction Mix | Nucleotide | 200 | mM | Variable | | Variable- as shown | |
| | complex | | | | | | |
| | water | | | 103.48 | μL | to 187.48 μL total | |
| Enzyme 1 | Pyrophosphatase | 2000 | U/mL | 0.2 | μL | 2 | U |
| Enzyme 2 | Phi29 | 1.6 | g/L | 0.32 | μL | 2.56 | mg/L |

DNA polymerase

### Sample Processing Procedure

To each aliquot, 800 μl (or 900 μL in later Examples—Examples 5 and 6) of water was added for dilution and then 500 μL of 25% PEG 8000 and 200 μL of 5M NaCl was added. Solutions were mixed by vigorously shaking and vortexing. The DNA was pelleted by centrifugation in a microcentrifuge (14,000×g, 30 minutes). The supernatants were carefully decanted, and the pellets were resuspended in 1800 μL of water (later examples use 1000 μL, 2500 μL or 5000 μL water—Examples 5 and 6) by positive-displacement pipetting and vigorous shaking, then left rotating overnight. Reaction DNA concentrations were quantified from UV absorption measurements on a nanodrop spectrophotometer the following day and then left for further rotary mixing.
Data is corrected for the 9× fold increase in reaction volume and concentrations are expressed in g/L of original volume vs dNTP concentrations used.

### Results

TABLE 6
DNA yields achieved at different concentrations
of the nucleotide complexes at 168 hours.

Raw Yield (g/L)

| | | | |
|---|---|---|---|
| Nucleotide | dNTP: | dNTP: | dNTP: |
| complex | 1Mg²⁺ | 4NH₄⁺ | 1Mg²⁺:2NH₄⁺ |

| | | | | |
|---|---|---|---|---|
| 20 | mM | 3.2 | 5.5 | 3.6 |
| 40 | mM | 6.9 | 11.1 | 7.5 |
| 60 | mM | 11.7 | 15.3 | 12.8 |
| 80 | mM | 15.6 | 17.3 | 17.0 |
| 100 | mM | 19.4 | 1.2 | 21.4 |
| 120 | mM | 24.7 | 0.8 | 25.7 |

TABLE 7
Efficiency of conversion of the nucleotide complexes
into DNA at different concentrations of the complexes

Incorporation Efficiency (%)

| | | | |
|---|---|---|---|
| Nucleotide | dNTP: | dNTP: | dNTP: |
| complex | 1Mg²⁺ | 4NH₄⁺ | 1Mg²⁺:2NH₄⁺ |

| | | | | |
|---|---|---|---|---|
| 20 | mM | 48.5% | 85.3% | 56.0% |
| 40 | mM | 52.8% | 85.1% | 57.4% |
| 60 | mM | 60.2% | 78.3% | 65.7% |
| 80 | mM | 60.0% | 66.7% | 65.3% |
| 100 | mM | 59.8% | 3.7% | 65.8% |
| 120 | mM | 63.3% | 2.1% | 66.0% |

This data leads the inventors to conclude that whilst initially reactions including 20-80 mM dNTPs with 4 monovalent counterions produce the greatest yield, once the concentration is raised to over 80 mM, dNTPs with 1 divalent counterion (in the case of the newly prepared nucleotides, postulated to be protonated or associated with hydronium ions to balance charge) there is an significant increase in raw DNA produced. By using prepared according to the present invention i.e. dNTP:1Mg²⁺ the level of dNTP usage increases. This highest yield of 24.7 g/L for the newly prepared nucleotide complexes compares to nucleotide complexes including 4NH₄⁺ which peak at a DNA yield of 17.3 g/L. It is noted that nucleotide complexes including a magnesium ion maintain a consistent efficiency of conversion to DNA throughout the range of dNTP concentrations tested.

### Example 3

Example 2 demonstrates that the newly prepared nucleotide complexes can be prepared and used for DNA synthesis in the absence of any additional buffering agent. The effect of a buffering agent was therefore investigated. Example 2 was therefore repeated in the presence of a buffer to determine if such was useful.
Reactions were set up at a 200 μL scale as follows: A Denaturation Mix was prepared and left at room temperature for 15 minutes while the Reaction Mix was assembled. These were then mixed, and the DNA polymerase and pyrophosphatase added. DNA amplification experiments were performed using various dNTPs at a range of dNTP concentrations, with the addition of 30 mM pH 8.0 Tris Buffer. Reactions were stopped after 168 hours, and samples were immediately processed as detailed below.
For the dNTP complexes lacking magnesium, an equimolar amount of magnesium salt, specified below, was added to the Reaction Mix. For the dNTPs complexes including magnesium a (i.e. 1Mg), as described above, no additional magnesium was supplied to the Reaction Mix. Table 8 shows the experimental protocol reaction setup for the dNTPs without magnesium (4NH₄*) while Table 9 shows 20 the reaction setup for the magnesium complexed dNTPs (1Mg²⁺, 1Mg²⁺:2NH₄⁺).
The Effects of Different Counterion Complexes, their Concentration and Buffer Addition on DNA Synthesis

TABLE 8
DNA synthesis by Rolling Circle Amplification (RCA) Reaction components for
examining the effects of various nucleotide complexes with added Buffer.

| | | Stock | | | | Final reaction | |
|---|---|---|---|---|---|---|---|
| | Reagent | concentration | | Volume | | concentration | |
| Denaturation | Plasmid | 1.393 | g/L | 0.29 | μL | 2 | ng/μL |
| Mix | Template | | | | | | |
| | NaOH | 1M | | 2.0 | μL | 10 | mM |
| | DNA primer | 5 | mM | 2.0 | μL | 50 | μM |
| | water | | | 7.71 | μL | to 12 μL | |
| Reaction Mix | Nucleotide | 200 | mM | Variable | | Variable- as shown | |
| | complex | | | | | | |
| | Tris pH 8.0 | 1M | | 6 | μL | 30 | mM |
| | MgCl₂ | 2000 | mM | Variable | | Equimolar to dNTP | |
| | | | | | | molar conc. | |
| | water | | | Variable | | to 187.48 μL total | |
| Enzyme 1 | Pyrophosphatase | 2000 | U/mL | 0.2 | μL | 2 | U |
| Enzyme 2 | Phi29 | 1.6 | g/L | 0.32 | μL | 2.56 | mg/L |

DNA polymerase

TABLE 9
DNA synthesis by Rolling Circle Amplification (RCA) Reaction components
for examining the effects of various nucleotide complexes with buffer.

| | | Stock | | Final reaction | |
|---|---|---|---|---|---|
| | Reagent | concentration | Volume | concentration | |
| Denaturation | Plasmid | 1.393 g/L | 0.29 μL | 2 | ng/μL |
| Mix | Template | | | | |
| | NaOH | 1M | 2.0 μL | 10 | mM |
| | DNA primer | 5 mM | 2.0 μL | 50 | μM |
| | water | | 7.71 μL | to 12 μL | |
| Reaction | Nucleotide | 200 mM | Variable | Variable- | |
| Mix | complex | | | as shown | |
| | Tris pH 8.0 | 1M | 6 μL | 30 | mM |
| | water | | Variable | to 187.48 μL total | |
| Enzyme 1 | Pyrophosphatase | 2000 U/ml | 0.2 μL | 2U | |
| Enzyme 2 | Phi29 | 1.6 g/L | 0.32 μL | 2.56 | mg/L |
| | DNA polymerase | | | | |

### Sample Processing Procedure

To each aliquot, 800 μL of water was added for dilution and then 500 μL of 25% PEG 8000 and 200 μL of 5M NaCl was added. Solutions were mixed by vigorously shaking and vortexing. The DNA was pelleted by centrifugation in a microcentrifuge (14,000×g, 30 minutes). The supernatants were carefully decanted, and the pellets were resuspended in 1800 μL of water by positive-displacement pipetting and vigorous shaking, then left rotating overnight. Reaction DNA concentrations were quantified from UV absorption measurements on a nanodrop spectrophotometer the following day and then left for further rotary mixing. Data is corrected for the 9× fold increase in reaction volume and concentrations are expressed in g/L of original volume vs dNTP concentrations used.

### Results

TABLE 10
DNA yields achieved at different concentrations
of the nucleotide complexes with buffer.

| | |
|---|---|
| Nucleotide | Raw DNA yield (g/L) |

| complex | dNTP: 1Mg²⁺ | dNTP: 4NH₄⁺ |
|---|---|---|
| 20 mM | 3.3 | 5.6 |
| 40 mM | 7.3 | 10.9 |
| 60 mM | 11.3 | 15.1 |
| 80 mM | 15.5 | 19.2 |
| 100 mM | 17.5 | 1.0 |
| 120 mM | 22.4 | 1.0 |

TABLE 11
Efficiency of conversion of the nucleotide complexes into DNA
at different concentrations of the complexes with buffer.

| | |
|---|---|
| Nucleotide | Incorporation Efficiency (%) |

| Complex | dNTP: 1Mg²⁺ | dNTP: 4NH₄⁺ |
|---|---|---|
| 20 mM | 50.8% | 85.5% |
| 40 mM | 56.1% | 84.0% |
| 60 mM | 58.0% | 77.3% |
| 80 mM | 59.8% | 73.7% |
| 100 mM | 53.9% | 3.1% |
| 120 mM | 57.5% | 2.6% |

This data suggests that that while initially 20-80 mM dNTPs the highest yields come from nucleotide complexes with 4 ammonium counterions, at higher concentrations, nucleotide complexes associated with 1 divalent counterion only (postulated to be protonated or associated with hydronium ions to balance charge) there is an significant increase in raw DNA produced. By using nucleotide complexes prepared according to the present invention i.e. dNTP:1Mg²⁺ the efficiency of nucleotide incorporation remains stable even at 100 mM. Standard dNTP:4NH₄⁺ have peak DNA yields of 19.2 g/L compared to 22.4 g/L using the nucleotides prepared according to the new method.
Nucleotide complexes prepared according to the present invention maintain their efficiency of incorporation into DNA throughout the range of nucleotide concentrations tested.
As seen here, the new nucleotide complexes did not perform better in a buffered environment, thus it is not a required component for these reduced-counterion complexes.

### Example 4

The experimental conditions set out in Example 2 were replicated for a different set of nucleotide complexes. dNTP:1Mg²⁺, dNTP:1Mg²⁺:2His, dNTP:4NH₄⁺
The Effects of Different Nucleotide Complexes and their Concentration on DNA Synthesis

TABLE 12
DNA synthesis by Rolling Circle Amplification (RCA) Reaction components
for examining the effects of various nucleotide complexes.

| | | Stock | | | Final reaction | |
|---|---|---|---|---|---|---|
| | Reagent | concentration | | Volume | concentration | |
| Denaturation | Plasmid | 595 | ng/μL | 0.34 μL | 1 | ng/μL |
| Mix | Template | | | | | |
| | NaOH | 1M | | 1.0 μL | 10 | mM |
| | DNA primer | 5 | mM | 1.0 μL | 50 | μM |
| | water | | | 3.66 μL | to 6.0 μL | |
| Reaction | Nucleotide | 200 | mM | Variable | Variable- | |
| Mix | complex | | | | as shown | |
| | MgCl₂ | 2000 | mM | Variable | Equimolar to dNTP | |
| | | | | | molar conc. | |
| | water | | | Variable | to 94.10 μL total | |
| Enzyme 1 | Pyrophosphatase | 2000 | U/mL | 0.10 μL | 1U | |
| Enzyme 2 | Phi29 | 0.80 | μg/μL | 0.30 μL | 2.4 | ng/μL |
| | DNA polymerase | | | | | |

TABLE 13
DNA synthesis by Rolling Circle Amplification (RCA) Reaction components
for examining the effects of the new nucleotide complexes.

| | | Stock | | | Final reaction | |
|---|---|---|---|---|---|---|
| | Reagent | concentration | | Volume | concentration | |
| Denaturation | Plasmid | 595 | ng/μL | 0.34 μL | 1 | ng/μL |
| Mix | Template | | | | | |
| | NaOH | 1M | | 1.0 μL | 10 | mM |
| | DNA primer | 5 | mM | 1.0 μL | 50 | μM |
| | water | | | 3.66 μL | to 6.0 μL | |
| Reaction | Nucleotide | 200 | mM | Variable | Variable- | |
| Mix | complex | | | | as shown | |
| | water | | | Variable | to 94.10 μL total | |
| Enzyme 1 | Pyrophosphatase | 2000 | U/mL | 0.10 μL | 1U | |
| Enzyme 2 | Phi29 | 0.80 | μg/μL | 0.30 μL | 2.4 | ng/μL |
| | DNA polymerase | | | | | |

### Sample Processing Procedure—as Example 2

### Results

TABLE 14
DNA yields achieved at different concentrations
of the nucleotide complexes. Post 48 hours

| | Raw Yield (g/L) | | |
|---|---|---|---|
| Nucleotide | dNTP: | dNTP: | dNTP: |
| complex | 1Mg²⁺ | 1Mg²⁺:2His | 4NH₄⁺ |
| 20 mM | 1.06 | 1.15 | 4.91 |
| 40 mM | 1.81 | 3.45 | 6.70 |
| 60 mM | 8.96 | 11.39 | 6.47 |
| 80 mM | 8.45 | 8.60 | 0.55 |
| 100 mM | 6.68 | 9.63 | 0.94 |
| 120 mM | 7.49 | 13.49 | 1.41 |

TABLE 15
DNA yields achieved at different concentrations
of the nucleotide complexes. Post 96 hours

| | Raw Yield (g/L) | | |
|---|---|---|---|
| Nucleotide | dNTP: | dNTP: | dNTP: |
| complex | 1Mg²⁺ | 1Mg²⁺:2His | 4NH₄⁺ |
| 20 mM | 1.57 | 1.60 | 5.77 |
| 40 mM | 3.20 | 6.63 | 9.43 |
| 60 mM | 12.69 | 12.26 | 12.78 |
| 80 mM | 13.02 | 11.62 | 2.61 |
| 100 mM | 12.73 | 13.21 | 3.38 |
| 120 mM | 16.03 | 13.31 | 4.22 |

TABLE 16
DNA yields achieved at different concentrations
of the nucleotide complexes. Post 168 hours

| | Raw Yield (g/L) | | |
|---|---|---|---|
| Nucleotide | dNTP: | dNTP: | dNTP: |
| complex | 1Mg²⁺ | 1Mg²⁺:2His | 4NH₄⁺ |
| 20 mM | 2.90 | 2.10 | 5.46 |
| 40 mM | 6.04 | 3.01 | 9.49 |
| 60 mM | 14.45 | 12.68 | 13.91 |
| 80 mM | 16.37 | 12.56 | 1.71 |
| 100 mM | 16.70 | 16.99 | 7.19 |
| 120 mM | 20.81 | 14.45 | 6.11 |

TABLE 17
DNA yields achieved at different concentrations
of the nucleotide complexes. Post 216 hours

| | Raw Yield (g/L) | | |
|---|---|---|---|
| Nucleotide | dNTP: | dNTP: | dNTP: |
| complex | 1Mg²⁺ | 1Mg²⁺:2His | 4NH₄⁺ |
| 20 mM | 2.09 | 1.96 | 5.45 |
| 40 mM | 4.94 | 7.70 | 10.39 |
| 60 mM | 14.37 | 11.16 | 14.37 |
| 80 mM | 16.41 | 12.64 | 4.85 |
| 100 mM | 17.86 | 16.24 | 7.25 |
| 120 mM | 21.16 | 14.25 | 8.87 |

TABLE 18
DNA yields achieved at different concentrations
of the nucleotide complexes. Post 480 hours

| | Raw Yield (g/L) | | |
|---|---|---|---|
| Nucleotide | dNTP: | dNTP: | dNTP: |
| complex | 1Mg²⁺ | 1Mg²⁺:2His | 4NH₄⁺ |
| 20 mM | 2.48 | 3.24 | 5.46 |
| 40 mM | 8.53 | 7.83 | 10.33 |
| 60 mM | 15.32 | 13.28 | 14.83 |
| 80 mM | 18.64 | 12.41 | 1.98 |
| 100 mM | 20.52 | 16.90 | 1.80 |
| 120 mM | 23.74 | 14.87 | 3.41 |

This data leads the inventors to conclude that whilst initially reactions including 20-40 mM dNTPs with 4 monovalent counterions produce the greatest yield after 20 days, once the concentration is raised to over 60 mM, dNTPs with 1 divalent counterion (in the case of the newly prepared nucleotides, associated with hydronium ions to balance charge), the 1 divalent counterion dNTPs take over, where there is a significant increase in raw DNA produced. 1Mg2+:2H₃O⁺ produces the highest yield of raw DNA compared to all other dNTPs and conditions tested, 23.74 g/L compared to 1Mg²⁺:2His and 4NH₄⁺ which peak at a DNA yield of 16.99 g/L and 14.83 g/L. The inventors note that 1Mg²⁺:2His dNTPs outperforms all other dNTPs at concentrations above 60 mM in the first 48 hours. This observation leads inventors to conclude that 1Mg²⁺:2His accelerates the initial reaction resulting in a significant raw yield in the initial 48 hours of the RCA reaction.

### Example 5

Examples 2 and 4 demonstrates that the newly prepared nucleotide complexes can be prepared and used for DNA synthesis. According to the previous observed results inventors concluded that depending on the 1Mg²⁺ dNTPs complex the initial reaction speed could be accelerated. Example 4 was therefore repeated over 24, 48 and 72 hours.
Reactions were set up as described previously in Example 4. Reactions were stopped after 24 hours, 48 hours and 72 hours and samples were immediately processed as detailed below.
For the dNTP complexes lacking magnesium, an equimolar amount of magnesium salt, specified below, was added to the Reaction Mix, For the dNTPs complexes including magnesium a (i.e. 1Mg), no additional magnesium was supplied to the Reaction Mix as described previously in Example 2 and Example 4, Table 12 and Table 13, respectively. However we included additional 1Mg²⁺ dNTPs complexes; 1Mg²⁺: 1His and 1Mg²⁺:2 to investigate acceleration effects of 1Mg²⁺ dNTP complexes at higher dNTP complex concentrations; 80 mM, 100 mM and 120 mM.

### Sample Processing Procedure—as Example 2

### The Effects of Different Nucleotide Complexes at Higher Concentrations on DNA Synthesis in the Initial Days of Reaction.

TABLE 19
DNA yields achieved at 80 mM of different 1Mg²⁺ complexes
nucleotide complexes. Post 24 hours, 48 hours, 72 hours.

| | Raw Yield (g/L) | | |
|---|---|---|---|
| Day | 1 | 2 | 3 |
| 1Mg²⁺:2H₃O⁺ | 6.71 | 12.48 | 16.43 |
| 1Mg²⁺:1His | 9.03 | 11.24 | 11.31 |
| 1Mg²⁺:2His | 9.79 | 11.83 | 12.47 |
| 1Mg²⁺:2Ylide | 5.24 | 7.92 | 7.71 |
| 4NH₄⁺ | 7.84 | 11.12 | 14.34 |

TABLE 20
DNA yields achieved at 100 mM of different 1Mg²⁺ complexes
nucleotide complexes. Post 24 hours, 48 hours, 72 hours.

| | Raw Yield (g/L) | | |
|---|---|---|---|
| Day | 1 | 2 | 3 |
| 1Mg²⁺:2H₃O⁺ | 5.02 | 9.78 | 13.10 |
| 1Mg²⁺:1His | 9.44 | 11.99 | 13.07 |
| 1Mg²⁺:2His | 7.35 | 13.74 | 15.34 |
| 1Mg²⁺:2Ylide | 8.12 | 8.26 | 8.54 |
| 4NH₄⁺ | 1.78 | 2.18 | 1.91 |

TABLE 21
DNA yields achieved at 120 mM of different 1Mg²⁺ complexes
nucleotide complexes. Post 24 hours, 48 hours, 72 hours.

| | Raw Yield (g/L) | | |
|---|---|---|---|
| Day | 1 | 2 | 3 |
| 1Mg²⁺:2H₃O⁺ | 4.06 | 6.01 | 9.98 |
| 1Mg²⁺:1His | 10.75 | 12.35 | 14.58 |
| 1Mg²⁺:2His | 3.87 | 17.62 | 17.48 |
| 1Mg²⁺:2Ylide | 9.26 | 9.21 | 7.75 |
| 4NH₄⁺ | 1.99 | 1.88 | 1.45 |

This data suggests that that while using nucleotide complexes prepared according to the present invention i.e. dNTP:1Mg²⁺ 2H₃O⁺ at 80 mM in long incubation reactions produces higher raw DNA yield, 16.43 g/L. In higher concentrations, 100 mM and 120 mM the other 1Mg²⁺ dNTP complexes invented (1Mg²⁺: 1His, 1Mg²⁺:2His) outperform 4 monovalent counterion dNTPs and 1 divalent counterion (in the case of the newly prepared nucleotides, associated with hydronium ions to balance charge) in the first 72 hours of the reaction, with 1Mg²⁺:2His producing the highest yields of DNA at higher dNTP concentrations (100 mM and 120 mM) after 72 hours, 17.48 g/L and 15.34 g/L, respectively. 1Mg²: 1His followed closely behind by matching or surpassing 1Mg²⁺ 2H₃O⁺. 1Mg²⁺: 2His produced the highest yield of DNA in higher concentrations after only 48 hours.
Inventors conclude that 1 divalent counterion complexes accelerate the reaction, as these complexes outperform 1 divalent counterion alone and 4 monovalent counterion dNTPs in the initial 24 hours. This is observed with 1Mg²⁺: 1His, 1Mg²⁺:2His in 80 mM-120 mM concentrations of dNTPs.
The 1Mg²⁺: Yide dNTPs outperform 4 monovalent counterion dNTPs and 1 divalent counterion alone at higher dNTP concentrations 100 mM-120 mM in the first 24 hours.
The inventors conclude this data shown above reinforces previous data that 1 divalent counterion dNTP complexes, specifically 1Mg²: 1His, 1Mg²⁺:2His and 1Mg²⁺:2Ylide as accelerators in the initial days of the reaction, thus allowing inventors to achieve a higher yield of DNA production in a shorter period of time.

### Example 6

Example 5 demonstrates that 1 divalent 1Mg²⁺ dNTP complexes at higher dNTP concentrations can be used to accelerate the RCA reaction in the first 72 hours to produce a higher yield of DNA. According to the previous observed results inventors concluded at higher dNTPs concentrations, 1Mg²⁺ dNTPs complexes accelerate DNA production allowing high DNA yields in 72 hours. Using this conclusion, the inventors decided to investigate the ability 1Mg²⁺ dNTPs complexes to produce DNA at higher dNTP concentrations. Example 5 was therefore repeated at dNTP concentrations 80 mM-120 mM and stopped at 120 hours and 240 hours.
Reactions were set up as described previously in Examples 2 and 4. Reactions were stopped after 120 hours and 240 hours samples were immediately processed as detailed below. Magnesium (i.e. 1Mg) dNTP complexes were used where no additional magnesium was supplied to the Reaction Mix as described previously in Example 4, Table 13.

### Sample Processing Procedure—as Example 2

### The Effects of Different Nucleotide Complexes at Higher Concentrations on DNA Synthesis.

TABLE 22
DNA yields achieved at different concentrations of
1Mg²⁺ complexes nucleotide complexes. Post 120 hours.

| | Raw Yield (g/L) | | | |
|---|---|---|---|---|
| [dNTP] | 1Mg²⁺:2H₃O⁺ | 1Mg²⁺:2His | 1Mg²⁺:1His | 1Mg²⁺:2Ylide |
| 80 mM | 11.05 | 12.64 | 11.51 | 13.08 |
| 100 mM | 8.96 | 14.43 | 17.76 | 4.39 |
| 120 mM | 5.00 | 17.72 | 19.75 | 0.98 |

TABLE 23
DNA yields achieved at different concentrations of
1Mg²⁺ complexes nucleotide complexes. Post 240 hours.

| | Raw Yield (g/L) | | | |
|---|---|---|---|---|
| [dNTP] | 1Mg²⁺:2H₃O⁺ | 1Mg²⁺:2His | 1Mg²⁺:1His | 1Mg²⁺:2Ylide |
| 80 mM | 18.26 | 18.13 | 17.53 | 19.54 |
| 100 mM | 24.07 | 3.25 | 21.44 | 7.81 |
| 120 mM | 16.97 | 19.85 | 26.93 | 2.30 |

This data suggests that that while using 1Mg²⁺ nucleotide complexes prepared according to the present invention i.e. dNTP:1Mg²⁺:2H₃O⁺, 1Mg2+:1His, 1Mg²⁺:2His at high dNTP concentrations 80 mM-120 mM produces higher raw DNA yields in long incubation reactions.
In higher dNTP concentrations, 1Mg²⁺:2His out performs all other 1Mg²⁺ dNTP complexes invented, 1Mg²⁺:2His produced the highest yields of raw DNA at 120 hours and 240 hours, 19.75 g/L and 26.93 g/L. 1Mg²⁺:2Ylide dNTPs outperform other 1Mg²⁺ dNTP complexes at 80 mM dNTP concentrations as inventors observed the dNTP complex produced the higher raw DNA yields compared to other 1Mg2+ dNTP complexes at this specific dNTP concentration over 120 hours and 240 hours; 13.08 g/L and 19.54 g/L respectively.
The inventors conclude this data shown above reinforces previous data that 1 divalent counterion dNTP complexes, specifically 1Mg²⁺: 1His, 1Mg²⁺:2His and 1Mg²⁺:2Ylide as accelerators in the initial days of the reaction, thus allowing inventors to achieve a higher yield of DNA production in a shorter time span.

### CONCLUSION

Inventors conclude there is a significant advantage of using 1Mg²⁺ dNTP complexes to not only accelerate DNA production in the initial hours of the RCA reaction but also to boost overall raw DNA yield production especially at higher dNTP concentrations.

1. A nucleotide complex comprising a nucleotide associated with a divalent cation which is present at a ratio of 0.2-1.5 divalent cations per nucleotide and optionally further associated with a zwitterionic molecule which is present in an amount of less than or equal to 4 zwitterionic molecules per nucleotide.

2. The nucleotide complex of claim 1 which is in solution, optionally in water.

3. The nucleotide complex of claim 1, wherein:
(i) the divalent cation is a divalent metal cation, preferably selected from any one or more of calcium, magnesium or manganese;
(ii) the zwitterionic molecule, if present, is an amino acid or ylide, preferably selected from any one or more of histidine, lysine, arginine or dimethylsulfoxonium-(isobutanoyl)methylide;
(iii) the nucleotide complex comprises less than 0.5 monovalent cations per nucleotide; and/or
(iv) the concentration of additional ions associated with the nucleotide complex are less than 5%, 4%, 3%, 2% or 1% of the concentration of the nucleotide complex, or are entirely absent.

4. The nucleotide complex of claim 1, wherein
(i) the nucleotide is associated with 1 magnesium ion per nucleotide;
(ii) the nucleotide is associated with 1 magnesium ion and at least one histidine molecule per nucleotide; or
(iii) the nucleotide is associated with 1 magnesium ion and at least one per dimethylsulfoxonium-(isobutanoyl)methylide molecule per nucleotide.

5. (canceled)

6. The nucleotide complex of claim 1, wherein the nucleotide complex is a deoxynucleoside triphosphate (dNTP).

7. A cell-free process for the enzymatic synthesis of DNA in solution comprising obtaining the nucleotide complex as claimed in claim 1 and adding a nucleotidyltransferase.

8. The cell-free process as claimed in claim 7, wherein said nucleotide complex is obtained at a concentration of 40 mM to 160 mM, optionally 50 to 150 mM or 60 to 140 mM.

9. (canceled)

10. (canceled)

11. The cell-free process according to claim 7, wherein magnesium or manganese salts are added to the reaction mixture as a co-factor for the nucleotidyltransferase, such that the total ratio of the magnesium and/or manganese to nucleotide does not exceed 2:1.

12. The cell-free process according to claim 7, wherein said nucleotidyltransferase is a DNA polymerase, preferably a strand-displacing DNA polymerase, and said synthesis is isothermal DNA synthesis.

13. (canceled)

14. (canceled)

15. A process for the preparation of a nucleotide complex comprising:
(i) providing a starting nucleotide complex which comprises a nucleotide associated with a polyatomic monovalent cation, wherein the polyatomic moiety of the cation is volatisable;
(ii) applying any one or more of heat, a vacuum and/or a change in pH to the starting nucleotide complex to enable a proportion of the polyatomic moieties to vaporise; and
(iii) before, during or after step (ii) mixing the starting nucleotide complex with a second nucleotide complex, said second nucleotide complex comprises a nucleotide associated with a divalent cation.

16. The process according to claim 15, wherein the starting nucleotide complex comprises a nucleotide associated with a monovalent cation present at a ratio of between 0.2 and 4 polyatomic monovalent cations per nucleotide; and/or
wherein the second nucleotide complex comprises a nucleotide associated with a divalent cation present at a ratio of between 0.5 and 4 divalent cations per nucleotide;
and/or wherein the second nucleotide complex comprises a nucleotide associated with a divalent cation, and the divalent cation is calcium, magnesium or manganese.

17. (canceled)

18. The process of claim 15, wherein said process further comprises:
(iv) before, during or after step (ii) mixing the starting nucleotide mixture with a zwitterionic molecule.

19. The process of claim 15, wherein said suitable conditions are heat and/or vacuum, optionally wherein these conditions are applied in step (ii).

20. The process of claim 15, wherein said proportion of polyatomic moieties that are vaporised is at least 90%, 91%, 92%, 93%, 94%, 95%, 96%, 97%, 98% or 99% of those present in the starting material.

21. (canceled)

22. The process of claim 15, wherein the polyatomic monovalent cation is the conjugate acid of a base, wherein the base is a volatile polyatomic moiety.

23. The process of claim 15, wherein a deprotonated form of the polyatomic monovalent cation is a volatile polyatomic moiety, preferably wherein the resultant volatile polyatomic moiety has a boiling point lower than 100° C. at standard atmospheric pressure

24. The process of claim 15, wherein said polyatomic monovalent cation is an ammonium ion or an ionic derivative thereof.

25. (canceled)

26. (canceled)

27. The process of claim 15, wherein the nucleotide complex is in powder form.

28. The process of claim 18, wherein the zwitterionic molecule is any one or more of histidine, lysine, arginine or dimethylsulfoxonium-(isobutanoyl)methylide.

29. (canceled)

30. The process of claim 15, wherein step (ii) comprises:
applying heat to the nucleotide complex, such that the temperature of the complex is above 40° C., above 50° C., above 60° C., above 70° C., above 80° C., above 90° C. or above 100° C.; and/or
applying a vacuum created at a pressure of between 105 Pa and 100 Pa.

31. (canceled)
